SEQUENCE LISTING (21 sequences)

SEQ ID NO: 1 - DNA, 1001 bp, Homo sapiens
  misc_feature (501)..(501): Y is C or T
tgcctgaggattaaataagaaaatgagtataacgcattttatccaggacttggcacataataaatatcgataaatgtgtatggttttggttgttaaggggtctgggatgaaatctaggatgtctggaggaaagagtactgaaaagtactgccagcaatacaaaaacgcccaagggaaagagacctcaaataatctttattacactttcttggctggccctacccactttcctctgctggaagttgtacacaggaaaataaactagcagagaaggatatattcatgattgtttcctccaagcgtctcaacaacttttcccctgatcattttcattttttcctttctccccactttatcatgaatttctttacctgcaatccagggtgcactaaagagataaacttggctttcagcatcaggctcccatcatgtgggcagtggggcaaccagatgccattctgagtgctgcatcagagcgggtgagactcgggcatcatggcyctggcacagcccagactccggcgtgccaaggcccatgctgcgtgttcacgccgcttcctcttctgaagtctgctgtatccacagatttaggaaccagcttgatggaagggagtgggcactggggagttttggagcatcaatccttccaggccatctgtagctagctttatatagtgcagggtaaagggacagatcaggatggaatggtttgctttgtttgacttcaatgggagggagggggacagaaggagtgggaatgctaaaattgcctgggagagggtgatgccaatttccacccccccacttttctcctccactattttggaacaaaattaacctgtggtttatctggagaggagagctgagaggcagtcacatctggctatctgatggtcacaagacacagaggtgctagtgtgaacttgtggattccgagacaatagaagaggttgctattattgctgaataatagggagaaagataagcagcaggatgctgtt

SEQ ID NO: 2 - DNA, 1001 bp, Homo sapiens
  misc_feature (501)..(501): M is A or C
atatttctttggtgctcatcccataataaaacatgaagagaagagcatcatttacctgatgaagtgtggcatggcttccatagactgagggctccagaaaatccaatctgacataatcacatgatggtgctgccctgttattgcagctttaggtgggtgagaggcagtattgcaagggaaattttgggaacctcccataaaggaatccttagttccctcagactaccaggacaaattgacacaaatttgtcagcttaaaataacagacatgtattctcccacagttcttgagaacaaagactacacttaaggtttcatgcggattaattatttcttgggggctcagagagagaatctatgcttctcttccagcttctagtgttctccagcaattcttggcagttccttagtttgtggataaagcactccaggctctgcctccatttccacatggctttctgttgtgtgtgtctctgtgtctgtctctgcttcctcttcttmtaataacaacacacattggatcaggggcctctagtccagtatgacgtcaacttgattacatctgcaaagaccctgtttccaaataaagtcacattcacatgtgccaggcgttagggcttcaatgtatctttttcaaggaatcccattcttaagccgcgatgttgattgtatctttcactgtttagaaagcagtagagtccactgcggcaaacatcctctggggacaaatggtaaatgccatgtagcaactggaaaaaaaaaaaaaggaaagaaaatcttcccttctctctctttttgtttccaaacttgaatctctaaggaagattctgattaagatttttgggagctgatatattatgttgagccatataaaactgctgttttgtgaagctaaaaagtttacatatagacagtttcttgttattcaacctaacagcaagtaaactaagaggcagaaaattattacatatttagaacgtgaccatttttatgaccccaga

SEQ ID NO: 3 - DNA, 501 bp, Homo sapiens
  misc_feature (251)..(251): H is C or T
gctcaagcagtcctcccaccttgagtagctgggaccacacacatgtgttaccatgcctggctaatttattttttgtagagacagggtcttgctatgttgcccaggctgatcttgaacttctgggctccagtgatcctcccacctcagcctcccaaagtgctgggattataagcatgagcccctgcaccagccatgtgattgttagattgtgcaacttagatggtacagaaaagcaaaggaaggaaaaatahcaaccttgtcctgtcacccaggaggagggagccagagacagacacatggtaagcacagactttgaacagtgtcaaagcttggaataagctgagccttacacaaatgctaaagacaaaacaaaagaggaaaaatagttttacgtttcaaacagcaccgagaaagagaaatgatcaataagtggaagaggatatcaactgaacaatttggttttctccaactttcttcccagcacctgctctgttggcatgc

SEQ ID NO: 4 - DNA, 1001 bp, Homo sapiens
  misc_feature (501)..(501): M is A or C
actccttcctggcttccagagatattttctgagaggagcctgcatttctacagatggcataaaaaaatgaggcctgcagaacacaccattattcctgcgaggccctcagttcattagttgaagcagccaagtcttcacgagccgcaactgatcagcaaatgggaaatgagcaagagtataaattcccagcaaaccaaatcattaacatttgctgcctgcatcctggaaacacagtaaatcaaatgtgtcatcatttatcaagacaaaatatcattactaattgggattaatttgctgctgtgggattgctgacctttgctgaaagaaaataaaactgctcgttagctgtcttcatttaattttccttgaagtagggagaggggaaaaggcattctggcttagggaggagggaggaaaggtagggtggagtgttcttatcttcctaacactacacattttaaagtcacaacttgatgagagaacctgctcctatgctgttgmcaatccataatccagggttttctatttgtgataacaatgcctctggttgtcattgtctgtgatttaacactgtcttgggaaataataattaaaataattgccagggtaaaattatacctggtttcgtttataaataggaaaaggcaagcccattgcaactctaggatttatctttctgcactaacattaaaggtgattcaaaagaaataacacataagtctgagcatccccagtgattctacatctgcctggggtctacatggaaccattgactcaattattttgggtattaacttttactctagtaggtatcaatttttttttttagggcagatgaatatgtgtgctatgctaaatggctgtaaatgctggagtcaggattcttgtctttagactctaaggaaactgtggatcctgagatgtcaactgtggattcaaatcaacacattaaacaccactgatatttacattttgtaaaacactgcatacaaattttattt

SEQ ID NO: 5 - DNA, 1001 bp, Homo sapiens
  misc_feature (501)..(501): R is A or G
aactttctctactcacagatactacatgtgaagtctggaggtattaactgacttgtgcaagataacacagcaagttaggaacacaattttctcctcagcccatctctcctctaaacaatgttattgctgagaggatggagggtaagctttacataggaaaagaatgctaaccacccatggtaacttgtaacatgcagaacagtggtgcgtttactgaggcatttgaatgggaaattctataaggacagagatggccaaggaaagtcttaaaggatatatgggaatgaagttggactttaaaagaagaaagaaccagatccaaattcagtcctccagtgagagaatcacattaaaaaaggctatgcttgatgctcatcctggaacctggatggttataatgaaatctatgaagatctcaagaaagcatcttcattggtataattcgaaaccatgccagctgggcaggtaaatcatgtaaactgcatcactgctatcagaatrttttaaggaaatgaggaaaggctaggtatgtttctattcatggaaccaaggggatggctgtgataaagaagacttagtctttggagagaccaccaaacaaggtatgtctgggttctaaaaacttgagatattgatgaacacctctcagatgcaggagcagaaggtccaggcagatcctggagatgactctattcctggtgactgggatttaagtaacaagagcttaatccccaggaggatgttttgagtaaatagatcctggaaaacaaaggatactggaaggaaaacaaaggcaggtttaggcataaggatgtaagataggctggaaccaggatattaagaggttaattaatttccagaaataaaataatatcaggggtgttggcattcagacacatggtcagagcagggcccaggacatggctaacatggcacttgccggccagcctaaggatttgaggacaaggacaagattatctacaggtagagatggatgagtt

SEQ ID NO: 6 - DNA, 1001 bp, Homo sapiens
  misc_feature (501)..(501): K is G or T
agtacccttctcctggctgtggtccagctgccaggtgccaccgcagaccgcaggtgctggaggggagaggcacgtggccagaggcgcctggtcctgaccaatcttataggctgatcttttaataagaacatctgcctgttctttgggacttgtccagcaaataacaggaaacattttctaagaaataagtataaactccgatgagccatggcaaaagcagtttttttaagcttagtcaagttgaagcagcgggagtggaggaggatcagagaaacctctgtagttgtgattggttccttgtcgcctccactgcgtacccgctgagctgggctttgggcaggtgagcagcacagaggcttccctcctcgtccctccccacaccccaccatcgccacagctccctccgccgccggtggggcacccagatggggaggaagctgaggaggccactgttcacccaaactggttggtccttcgggcaatgctgggaatagggtgggkgctctgggcaagcccaggaggaggagggcctggctgagttccaggaacatctcacaccacaagggaggatctagctggtgccctccctgtactgtcaggaggccagagggcccttctccagctcatcagggagggaactgctctggcagggagtagggaaggctgtttgtggcatccctcaagaaagctcctgcctcagaagaggcccaggcaaaaagagcccacaacagtctacctgtggagcagggggagcccacagagcccagggggagcccacagagcccagtgagagctgggggtccttgctgccaactcagaccctcagatccaagccaatggctttggggcagggcagccaaagccatggtaagatagcctacaccacagagccccagaggcaggggctgccccgggctggcacatttggaaaggatgcacttgcaccctgtccacggaagagctgtcatcaggcctcaagcatctcagaatcctccccaggc

SEQ ID NO: 7 - DNA, 1001 bp, Homo sapiens
  misc_feature (501)..(501): Y is C or T
ttgctgggaaggggaaatcaaggtgcaggttctcttggaaggaggcaggatcacaagcgggtcctggggtggagagggggaaggtgtgagtggaaagagcaagtgtgcgtgacacaaccggcacagtcggcctctttctccactggatgggaactccctaaggcagagggctttgtgttttgtacattgttgtgtccccagcacctagaacagagcctgatacattgtaggagctcagtaaatatttattggatgagttgtaatatttattgtaaatattagtaaatatttattgagttgagttggtctttccagtgaagtggttgcttctctgtgaagctctgtgggaaacataaatggtgggagataggttacctgtcgttagggctggccatatgtgagccctataaccatttggtattcattcattcattcattcattcattcattcatggaatatttgcccagcttctattctgtaatatattgtattagtcactyggggcacaaatatgaaagccaacacatatttcttcaggacaggtgcagtggctcacacctgtagtcccagcactttgggaggctgaggcaggaggatcccttgaggtcaggagttcaagactagcctgggtgacatagcgagaccttgtctctacaaaaaattaaaaaataaatccagacagagcagtgtacctgtagtcttagctacttgggaggctgaggtgggaggatcgcttgagcccaagacttggaggctgcagtgagctatgattgtgccactgcactccagcctgggtgacagagtgagaccctattgttaaaaaattaattaattaattaaaaatatttctttaatacccatcatgagccaggctctgggggataccatggtgcctcaggctggaaacaccttctttggtagaagacacagactgtctaagccacagacgggggcgcgtcggtctctctctggaagtggagatgctgggaaaggctttgca

SEQ ID NO: 8 - DNA, 501 bp, Homo sapiens
  misc_feature (251)..(251): Y is C or T
tttcaccttgttcttaactcttttccatgacactgtgtatggcttttaagcgagtccagaaaaacggctatcattactacttaatataaaccatggggttctgtatcatgtaaacccagagcaactcctctttgaagcaccgacattcaaggctctacataacatgtttcaactctatcttagtgctatacattttccaagtacctgcgaggaaacagatagcagcacctccatttttctgtaaattgtgyttttgttgctgttctaagactccttccaaggctgttaaactttgaagagagttgggaaaagacaagaacatcttgccttcccgattgggtattcaggcttcactcagtaaagctgtcatgaagtagcgcaaagggtcagagacaaaccacatggcatctgatgtttgtctgtataagtttaaaataaaaatgtgacactacaagaaggttaacatgtggacctaattttgaaaatacaaacaggaagcac

SEQ ID NO: 9 - DNA, 1001 bp, Homo sapiens
  misc_feature (501)..(501): R is A or G
ttgaatataaatgtcagacattactgaaaactgtttcataatgtgcttatgcatcaatttcattgttttactcttttattgctatagaagcaggcagatatacgagtgtgtgtatacatatatttgtatacatatgtgtgtgtattgtgtgtatgtaggtgtgtaattaatagacaagtataacaactagtaattaacaaataaataacatcaattatttacctgttttaacatcacctggccacagttccacctaaatgatggcaacatgtttccaatctgtggctttctgctacgttttatttgccttggaatatttgcatttactcatcaatgctacaaacttacttacatattgtaggggcatgcagatgtattccatgagcttcaattaataaatgattgggactaaaaaggaaaaagtaaagaaattgaaatttgacctactgcccaaagcttgctaattgcttattttatttcagttttaatcttcaggtcctragcaaattatccatcataaccagctgatatggtttggctgtgccctcactgaaatctcaaattgagttatatctgtcagaattcccaagtgttgtgggagcgacctgggggaggtaattgagtcaggggggctggtctttaccgtgctatcatcatgatagtaaataagtctcaagagatctgatgggtttaacagaggtttctgcttttgcttctttcatatttttctcttgccaccaccatgtaagaagtgcctttcacctcccaccatgattttgaggcctccccaggcaagcaaaactgtaaggacaattaaaactctttttgtttccagtttcaggtatgtctttatcagcagcatgaaaatgaactaatacagttaattggtaccaggagtggtgtgatgctgaaaagacacctgaaaatgtgcaagaggctttggaactgggtaacaggcaaaggttctaacagtttggagggctcagaagaagacaggaaaa

SEQ ID NO: 10 - DNA, 1001 bp, Homo sapiens
  misc_feature (501)..(501): R is A or G
ccttacttttttccttccatctcttttcttctctttactggcatttatttattaaacatttgcatagcacttaatatgtagtaagcactgttttaagcactgtaaagtattaatttatttaattctaatagcaaccctatgagataggtattatttatatcatccacattttaaatctgagatggaaacattgacttaagaaacatgtccaaggccatggagcttgcatttggactcaggcatctggtcccagaatctgtgcttctcccactatgatattctgcttcttatttattctgctctccctgaagatgcacataatggaaatgaggtctcttcactgcatctctccatattgatttatgttctccagtttaaatttaattttgggggtgcttaccccagtgagttagcctgctttggtgcagagacaaaccaacctcgaatctccgtagctgactgcgaacacgtatttcttggccacttacatgccagcagtcrtaaatcacccgtggaggctctgttctatttctcttctcattctggcgggaatgtataaccagcttaccaggaagggagctaatacatggaaaccatcatacaatttgccacgaccattaactaagcccttggtacatctgtacagtcctctcttggccgttacatgtaatagcaatgctctgaggccaaaggttcaacttctgttcacaaaaagaagacaaaaagctgattcagagtaactcaaatatttttgtatacataaaatgaatgaagttttggctggtactttttaacactttggtggagatgatacaggtaaaaatccagtaacaggattcttctctgcatcatcagattccttttttactcctaagggttaaaaaaactttcacatggttataaattctctgaacccataattttttttctctagcagcttcttcaacataattttgctcagttcagtttatcaataatttttcaaatgcctgctgtggagc

SEQ ID NO: 11 - DNA, 1001 bp, Homo sapiens
  misc_feature (501)..(501): Y is C or T
caaaaagattagaactcactgaaggctccgatgatccttagcatttcttacctataaaatattttttaattaaggtattcacactgtttttttttagatgcaatactactgcacacttactatagtataaacacaacttccgtatgcattggttaacgaaaaaattggtatgacttgcttttttgcaatcttcactttagttaggtgatctgcaatatctctgaggtatgcctgtattgaataattgcattgactaaagaatgtttctcccccatacttcctcatttaaatgtaaaggcatttgattattgtttgttcatattatttaccctggaaatggcatttaacttaagtcaataaactatatctgcttgtgtatctcttggaacgactccatgtaccccaaggatagatgagatctaataatcattaacatgtcatattaattctgaaaaagttacagtaaaaagtttatttgcaaagctcttaggaaaagcacaygcctacatatacaaaagagagtaaaccacccccaatgcaaaatcaaaactaaaaatgataaaaaatccattatctttatcattatggagtagtggggtgagctgggtggagaaacagatgttaacaaaaataaaagctaggcatgcataaatgagagctggtcatactcgtgtattttgtatgtttgaattttttcacaataaaaaattaaaggaaaaatatttatttgctaataatttcccatactaatttgtaagattttccttttgagtatttttctttcccatttgaaacagtaattgatttttagttcatatatcagtggatatcttctattgctagaatgaactcaggactaattctcttccgatttttaatttttacagaggtgtgaattgcttataatagacaagtagattgaaattttaaaagttttattatagcaatgttactcagttctttatatgcctccagctcattacatgccaaaagtcatacag

SEQ ID NO: 12 - DNA, 401 bp, Homo sapiens
  misc_feature (201)..(201): R is A or G
tcctcccgcctcggcctcccaaagtactgggattacaggcgtgagccaccatgcccagcccagttaggaatttcataaacacttcccatggttaccttgctgatgtgcagcatcaaggaaaaaaatctcatttacaatttcatcaagccagctactcatatgggtgttttcccacctgaacactaagaaattcaactcacrgtaaaaacttattttctatataactgtgttctacataaacttaattcagttatatgctgatttgctatcttcaactgtatatatttatttaaaaataaaaagtcatttctgctcacacaattttccttcacggtaaacagaatgtgcttttggtaataagctagcttggttttagcaatgttgttgttttgaacatttgg

SEQ ID NO: 13 - DNA, 1001 bp, Homo sapiens
  misc_feature (501)..(501): K is G or T
aaactccatttcatttagctgcatttaaataaagatgaattatagcttcactgcaagatacaggaagtagccaaatttttaagtgtattttacaaaagatgcctatgaagaaatcaatatcacaaacctgaatactcgattcaagctctggcagctttcttatttaactcccagttgatttgttttcacagagtaaattaccagaagggcatttgtctcattctctctttactaaaaggtctttaacttctatttgcaggtgtttttgccttctaatctttctcttcattgtttctccagattttgtcacgataacagatgtactttttgtattctttcaaatcctgccattttgcttaagaagaaaaaagatttaaatcaaattgtggttcatttgtgatactctccacagacatttataactgacaactgaatgcatcagttactttaaacagcctgactacagtcattcataacaaaacaaagaggagtgactagcakaggactaactgatgagcagttctgtggagttgaggaaggttgtaatactttacatctacaacttaagatttcacagaagtctctgagacatatttacattacagattatgattcaaattttaaagcacaaggcaacattgtttggcccagctacttgattatgacttcttttcaatgtgctgttcccctgtaaacatcatggttgacgtttcaggcatttgcttcttctctctaccaaggacttgttttcattaacattgatgaacagctttcattttatctgtgtgtattctacaaaaagcaaaccttgtttgaaatttgtttcatattggaagaatttacttcatctcatagtcacagtgggcctttagctgccaatttaaaatgaagagacactttgcaaagtgtaagttattttattttaatgacattttatactcacgattcacatttttgtgcaggtaaaataggatatcattaagatcaccatcattgtgata

SEQ ID NO: 14 - DNA, 942 bp, Homo sapiens
  misc_feature (615)..(615): Y is C or T
atatcaacataatccaaatgagtgaagttaactcagtttttaatgtctagtaataacttataacataattctcaaagtatagtccaggaaccctagtggttcttgagaccctttcaggaggtctgcaaggtctcacatgcatagttttccagaagctagtgaaaagtgatgtccatagcagactgaatacagaagcatatatgtgaatccggctattttctgttaagctagacattaaagacatttgtaaaaatgtaaaattttctacttttttcagaaaatagttatttttcataaaacattatttgtattaagatataatgggtttattactgctatttttaaaaaataataaatattattacattttattttaatttcaaatacaataaatgtcaatgaataaaactcacattaacaaaagccctttggggaccaaaggattgtaaaggggtcatgacatcaaaatgtttaaaaatcaccagcttcggacatatctaatcctttactagatttaactacaaataagtatataaaattcagaataaataaggtgttcaaaatgctatgaacaccttcattgattaaaaaattatgtggggaaagtcaggaagygggaaaacaatatggtgttatagaaaggacaatacttttatatacagaaatcctgggttcaaatgtactagacatttccctgagtaagttactgaaccctcttaagcttcaacctctttgcctataaaatagagacactggcaaactgccttaccttcttctccaggttaagagagtcaagtgagataatgactataaaacgctatttcaatacattatattgaatattgacaatgaaaaaaaaaaccactacatcactacaaacataaaatcatagctattattaaagtttacctctgtgacagacagatggacacttatggtttc

SEQ ID NO: 15 - DNA, 801 bp, Homo sapiens
  misc_feature (401)..(401): K is G or T
caaagaaacttgccaggatcacaaagctaacttttaaaagttaaaacagtcttctgacttccaatgttctttctaccacatttttctatatttcttgattttcctcatcttcataattttaattttaaaagtttatttatttcaatacagttgcattacaattccaagacgctaaagactgaatcacctaatgcctccaggggatcttccttgccaagaacactgtccaaagagtccaagctgtatggtatgaaagatagtgcaacatctcctccttctcctcctttaccttccactgtccagagcaagactaacaccttacttcctccccagcccccacctattcctgcaggtatttaaggagtatatactttattttttgtcattttaaaaatttttakttattacctacttgctcttttgagactttccaatgcacatgtttaagaaaaaaggacatacatttgtcaataatgactctacagacttcatatatggaagtgtgggccaaccattttcacctgtttttgaaattgacattactttagaatatctgttataaaataaataatataccaaatagtttacgtaattaaatttttaggtctgtatatataaaaagagtagttttatgtagctcaaatttgggagcttatgagcagatggaaatatcaaacttgagagtacattgggtttaaccacatttattacttctctgttgtataacacattacccagaatttagtggcttacaacctcaaccacaggttctgctgatcagaaatgtaggcacagtttggc

SEQ ID NO: 16 - DNA, 1001 bp, Homo sapiens
  misc_feature (501)..(501): R is A or G
ttacttaacaacgttgagctagagaatgctgccacgtagaggattgtactcagggacacggtaacagtaaactggaattgtggaaggcagctaacttatggcaaataagtagtaaccctagatttcccaggattcctgtggcttggctaatttgaatcattttgtggctctgagacataataggtgtcccttttgtttgggatctggtcccagggccactagggcaggtgcatagtggcctggaatgcaagagcctcataacagaagtgtttggggtgtgtatttaatcaactgctcaagataggaaactgataagcttgtagccaggccttacaactgggttaagatggcacttcaaaaaacaaaatgaaacaaaatatattataattaactccttgatggcttgacatcagttttgagcttgattatttggggtatttgagtggcccaagtagtagaggaaatttttacggtgtaggacttaagttaccctaaataacrtagaaagaattttgttatgagagtagaaaggaacgaaaagtctaaggactccctgtaggctggccccatatctaaggtcccatatatatggcattagtcaagaaaacaaatttaaaatgcttagagtctctaacaatattttggtgaggttgtaaagtgtgttacagcattagttatttatgacaataatattttggtgtacttatcgagtcaattgaaggtggcaggtccaacagttatattactggattatatggactcataattctgtaaggtatttaaaagtgagtagtatttgtttctgaccaagtggtagtcatggttacagacacacagttcgagcttgtggtggcacatcagaaggggaagtaggaaaacgctctgggggtaggaagtctagttaaatggtacaccagtatgaggcagttacctactgaagcaagcaataatctccattaatttttagaatggcaattatagtagtaacctccacaagattc

SEQ ID NO: 17 - DNA, 1001 bp, Homo sapiens
  misc_feature (501)..(501): R is A or G
tgcagtctattaaggaggaagaaaagaaaggaagtagcattcactgatttggggtaccaagtttaatgctacaaacttcatacatgctgtgtttaaacagtaagaaaacgtaaaacaaatgtcattctcctcattttatacatgaagaaactgaagcatagagctattatataagtaaattaaaaagtcaggagttgactccaggtctatctgactccaaggcctgtgtttctcttttatcatcccaggtttctcaaaaggtggcccacagctcacttctatcacagtaatttgaggtgcctgagcctcacaccttgcttactgaattagaatatccagggctggagtataggaatctgcagttttaacaagttcccaagctgactctatactatagcggatcatgctgtctctgcaacacaacagtgaaattctaaacaatctctttaaacaaactagaaaatgtacgaaaaagaagtaagattcagtaggcttaagagrgggggccttaaggcaagtggaaaatcctacagaacaaggagtttgaggctggggaattcatcctaagccagttagtttcctgggtaatcttgggcaagtcacctaacttcactcaatctgtccccaaaaaaagtctacattttctccatagtaaaatgttgcaatcaggctaaataatctctaaagccttttcaaaagttatttgcaaagatttaaagagctttaaactgagacaataaaacacatacaggatgaagaaagtattaacatttaaattaacagataatcctagcaagattttttgtagatatacacaggcttcttctaaaatttatatggaaaggaaacagaactaaaaagttaaaacaattttgaaaaagaagaataaagtcagaggaatcacaatacccagttttaagacttactgtgtaactatagtaatcaacacagcatggtatttgcagagaaaaaggtccatagatcaatggaacagattacag

SEQ ID NO: 18 - DNA, 1114 bp, Homo sapiens
  misc_feature (201)..(201): S is C or G
cctctcactcagactcccttcagtgtctgcaactataaagtagttttccttccagctgagctggtataagaggaggctttccctcctatttctagtttacaacagccacttaagaaagaagtggcttttttctatcggtaaaatttcagacacctagcaggtgatgatggagatatttggggacaaaagagggcactggastctaaaacagaaaagcatagttacttattgctatggttcaactgtctacgtcctccaaaatccatgtgttggaacctaaaccccaaagtgacgctattaaaaggtggcgccttttaggaagtgatgaagtcctgagggctctgccttcgtgaatggattagtgctctgataaaagaggcagatgagagcacttcagtgtactttttttctttttttttaattgagacagagtctggctctgtcacccaggatggagtgcagtggcacgatctcagttcactgaaacctccacctcccaggttcaagcaattctcctgcctcagcctcctgagtagctgggattataggcacatgccaccatgcccagctaattttttttttttttttttttttgagacagagtctcactctgtcgcccaggctggagtgctaagctccgcctcctgggttcacgtcattctcctgcctcagtctcccgagtagctgggactacaggcgcccagatggtttcgatctcctgatctcgtgatccacccaccttggcctcccaaagtgctgggattacaggaatgagccacaggacccggcttcaggtcacagcaagaaggtccttgcaagacactgaatgctggtaccttgattttgggctgcctagcctccagaactataaaagataatttatgttccttaaaattacccagtctcaggcatttgttatagcagcacaaatggactgagacactacaccaagtcattagcccagactcatttttagagcttcaggctcgagatgtattagaattagactctagccacagttagttttggaggaaatggccctgcgtaagtccattcctcccccagatggaaggatatgagactgagtccttctgtcttttagatccagaaatca

SEQ ID NO: 19 - DNA, 456 bp, Homo sapiens
  misc_feature (256)..(256): K is G or T
ttccaccagcagtgtgtgggggttctgatttctccacattttcaccaacacttggtattacctgtcttttcaattttagcaatccttgaggatgtgaagtggtatttcataatgattttttgatacatatttttaaaagagccaatgaatatggatcaaaaacctcattcgacatgtgtcatgaggttttgctctgccactctctggttatcctgtgaggatgtctccctgacctctcccagaacaaaggaaagakcaaaggcaattccttgaaatgtagcgggctggtagggggtttgccctggcacagatgtgtccttcaggctctgactgtgaagaacaggctctgtcattgcacaaagacagtggctttgcccagatggtgtcttgctgtatgtgcaggagcattacaatgtagaaatactggaaacattaatggaaaggtataagaggggc

SEQ ID NO: 20 - DNA, 1001 bp, Homo sapiens
  misc_feature (501)..(501): R is A or G
ttgggaaagacttgactaagagacaaaacctagacaagagacaagtcagagcttcggctatcaataaaaaggaagacattttgcaataggagtatatcagtatggaacataaaaagattcattcttgtacctaagttagccatagtgtatattaataaatggatttgagccttcccctgaatcagaaaaatttgaaaaggtgtctaggtaattcagacagtttcatttattcatctattcatccttccatccatccatcaattcattcatctgtccacctattcatgcatccatattttatccattcatgcatccatatttttgttgtttaacaattacagcaggggcttttaacgagggctctgatgatgggtacaatcatctaaatttgattcatccttgcattcctagtgttttcctttgctaccgatacctgccctgtgtaaactatttgctaaataaagtaactagtgctcttaattacagctaggtaaacccagrtatgaataagtttgctttttatatgattgcttaatggccatgtgtaggatttatcctatctacatttgagaaattaacataccagaggggaccatgactagtttaaccatgtctagaagacacagctttagctgtttgacaaagtgaatccatctccttcccattctaacacaccactagaaagggctaaggccagattcatgcattaggagctaatcagtcagatctcaggattcagtagatccaattatttttgtttaacaccacttgaagaggtcaccacagagacccttaatcaaactgactgagtttgggataactgcatttatgggaacaggactccgacttgttcatataaccggtgccagttgaggaggatcgctctgtgagtttggtcgatacagtcaacttctttagacaactcaaattgaaatcttttcatttcttcaaaaacagagtaagtcctttaaactttaacctagaagcaacacagcgtgtat

SEQ ID NO: 21 - DNA, 501 bp, Homo sapiens
  misc_feature (251)..(251): R is A or G
cagtctccaattcttaactacagtataggccaaacactgcagaggcatgtcctccacaaattcttatgttgaagtcctaatgcccaattcctcaaaatgtatccttacttggtcactgcagatgtagtaagttaagatgaggttatatggagtagagtgagcagctaattcaatataactgtgtccttataaaaaggggaaacttggacaaaggcttacaacacagggagaattccacatgccatgtgaargcagagatctacaagccaaggaacatcaaagactgccagcaaaccaaaaaaaaaaaaagccaggggagaggcatgatacagattctctctcaccacccttagaaggaaccaaccctgctgacaccttcatcccagacttccagcctctagaattcagagataagaaatcactgggtttaagccactcggtctgtggtactttgttataaagccctggcaaactaataaacttacttacctatagttgtct

(19) United States
(12) Patent Application Publication
(10) Pub. No.: US 2017/0240965 A1
(43) Pub. Date: Aug. 24, 2017

(54) METHODS FOR PROGNOSING HEART TRANSPLANT

(71) Applicant: Cedars-Sinai Medical Center, Los Angeles, CA (US)

(72) Inventors: Bernice Coleman, Ladera Heights, CA (US); Mark O. Goodarzi, Los Angeles, CA (US); Jon Kobashigawa, Playa Del Rey, CA (US)

(73) Assignee: Cedars-Sinai Medical Center, Los Angeles, CA (US)

(21) Appl. No.: 15/439,830

(22) Filed: Feb. 22, 2017

Related U.S. Application Data
(60) Provisional application No. 62/298,338, filed on Feb. 22, 2016.

(51) Int. Cl. C12Q 1/68

(52) U.S. Cl. CPC C12Q 1/6881; C12Q 2600/172; C12Q 2600/156; C12Q 2600/106

(57) ABSTRACT

Provided herein is a method, comprising: obtaining a sample from the subject; assaying the sample to detect the risk alleles at one or more SNPs; calculating a genetic risk score (GRS) of the subject based on the detected risk alleles at the one or more SNPs; determining that the subject has an increased likelihood of poor prognosis if the GRS of the subject is above the mean or median GRS of the sample population or determining that the subject has an increased likelihood of good prognosis if the GRS of the subject is the same as or above the mean or median GRS of the sample population; and selecting a therapy if poor prognosis is determined.

### CROSS-REFERENCE TO RELATED APPLICATIONS

This application claims benefit of priority under 35 U.S.C. §119(e) of U.S. Provisional Patent Application No. 62/298,338 filed on Feb. 22, 2016, the contents of which are incorporated by reference in their entirety.

### STATEMENT REGARDING FEDERALLY SPONSORED RESEARCH OR DEVELOPMENT

This invention was made with government support under Grant No. TR000124 awarded by National Institutes of Health. The government has certain rights in the invention.

### FIELD OF THE INVENTION

The invention relates to genetics, medicine and heart transplantation.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Of the 570,000 cases of Stage D advanced heart failure unresponsive to maximum medical management, only 4,000 are potential candidates for heart transplantation (Htx). Ultimately, matching the appropriate precious donor organ to the recipient is the onerous charge of each individual heart transplant program. In 2014, the majority of the 2,174 heart transplants performed in the United States were implanted into 1420 Caucasian American and 445 African American recipients. However, Htx outcomes are dismal in African American (AfAm) compared to Caucasian American (CaAm) recipients. When exploring cause of death post heart transplantation, AfAm were more likely to die of cardiovascular events including graft failure compared to [the fewer unlucky] CaAm who more commonly died of infection or cancer. Retrospective large scale investigations using the United Network Organ Sharing (UNOS) national database also demonstrate worse survival for AfAm Htx recipients. Allen et al, found a 46% increase in cumulative risk of death at 10 years when comparing African Americans to Caucasian American heart transplant recipients. Analyzing survival by eras between 1987 and 2008, African Americans at 6 months and 6 years had significantly higher mortality when compared to Caucasian or Hispanic heart transplant recipients.

As such, for an informed clinical decision, there still exists a great need for methods, compositions and kits that can categorize/classify/stratify/subtype those patients who plan to receive or have received a heart transplant, and methods, compositions and kits that can prognose and/or treat them.

### SUMMARY

Provided herein is a method, comprising: obtaining a sample from the subject; assaying the sample to detect the risk alleles at one or more SNPs; calculating a genetic risk score (GRS) of the subject based on the detected risk alleles at the one or more SNPs; determining that the subject has an increased likelihood of poor prognosis if the GRS of the subject is above the mean or median GRS of the sample population or determining that the subject has an increased likelihood of good prognosis if the GRS of the subject is the same as or above the mean or median GRS of the sample population; and selecting a therapy if poor prognosis is determined.

In some embodiments, the subject has end-stage heart failure or a severe coronary artery disease.

In some embodiments, the subject is waiting for a heart transplant or has received a heart transplant.

In some embodiments, the GRS is the total number of the detected risk alleles at the one or more SNPs.

In some embodiments, the subject is of African ancestry and the one or more SNPs comprise one, two, three, four, five, six, or more, or all of: rs12030062 (SEQ ID NO: 1), rs2727438 (SEQ ID NO: 2), rs73266737 (SEQ ID NO: 3), rs8032616 (SEQ ID NO: 4), rs10519060 (SEQ ID NO: 5), rs3785437 (SEQ ID NO: 6), rs7221109 (SEQ ID NO: 7), rs62076937 (SEQ ID NO: 8), and rs2826929 (SEQ ID NO: 9).

In some embodiments, the GRS of the subject with African is above the median or mean GRS of the population of African ancestry and the heart transplant is prognosed with a poor clinical outcome.

In some embodiments, the subject is prognosed with a poor heart transplant and the selected therapies comprise heart transplant and administration of an effective amount of an IL-6 inhibitor, a JAK-STAT inhibitor or combinations thereof, wherein the inhibitors are administered before, during and/or after the heart transplant.

In some embodiments, the subject is prognosed with a good heart transplant and the selected therapies comprise heart transplant.

In some embodiments, the subject is of European ancestry and the one or more SNPs comprise one, two, three, four, five, six, or more, or all of: rs6690278 (SEQ ID NO: 10), rs2355570 (SEQ ID NO: 11), rs115230839 (SEQ ID NO: 12), rs17050452 (SEQ ID NO: 13), rs7688988 (SEQ ID NO: 14), rs80165265 (SEQ ID NO: 15), rs1991764 (SEQ ID NO: 16), rs4922070 (SEQ ID NO: 17), rs7957672 (SEQ ID NO: 18), rs2544081 (SEQ ID NO: 19), rs6564724 (SEQ ID NO: 20), and rs111315210 (SEQ ID NO: 21).

In some embodiments, the GRS of the subject is above the median or mean GRS of the population of European ancestry, and the heart transplant is prognosed with a poor clinical outcome.

Also provided herein is a method of classifying a subject with a cardiovascular condition, comprising: obtaining a sample from the subject; assaying the sample to detect the risk alleles at one or more SNPs; calculating a genetic risk score (GRS) of the subject based on the detected risk alleles at the one or more SNPs; and classifying the subject into a group based on the GRS of the subject. In some embodiments, the cardiovascular condition is end-stage heart failure or a severe coronary artery disease. In some embodiments, the GRS of the subject is not above the median or mean GRS of the population of the same ancestry as the subject, and the subject is classified into a low risk group. In some embodiments, the GRS of the subject is above the median or mean GRS of the population of the same ancestry as the subject, and the subject is classified into a high risk group.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with various embodiments of the invention, impact of combined IL-6 GG -174/TNFα GG -308 genotypes on survival for both ethnic/racial heart transplant groups.

FIG. 2 depicts, in accordance with various embodiments of the invention, IFNγ AA (+874) survival for both ethnic/racial heart transplant groups.

FIG. 3 depicts, in accordance with various embodiments of the invention, ten year survival by ethnic/racial low risk and high risk GRS groups.

FIG. 4 depicts, in accordance with various embodiments of the invention, Manhattan Plot African American Signal: Chromosomes 1, 3, 10, 15 and 19.

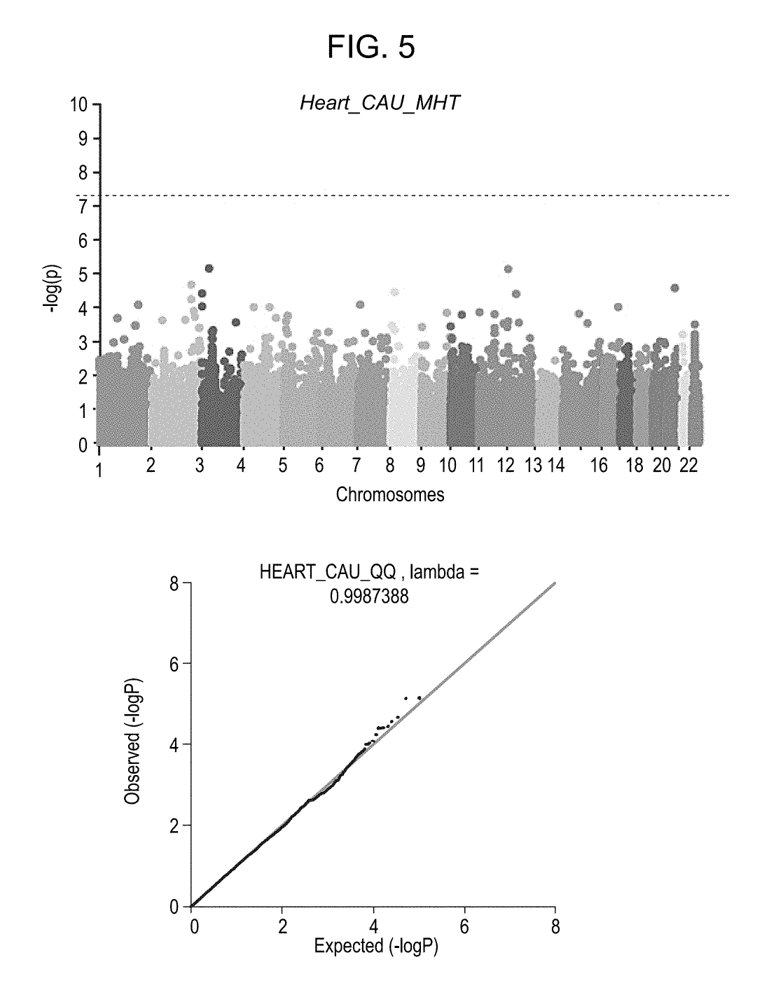

FIG. 5 depicts, in accordance with various embodiments of the invention, Manhattan Plot Caucasian American Signal: Chromosomes 2, 3, 8, 12, 14 and 20.

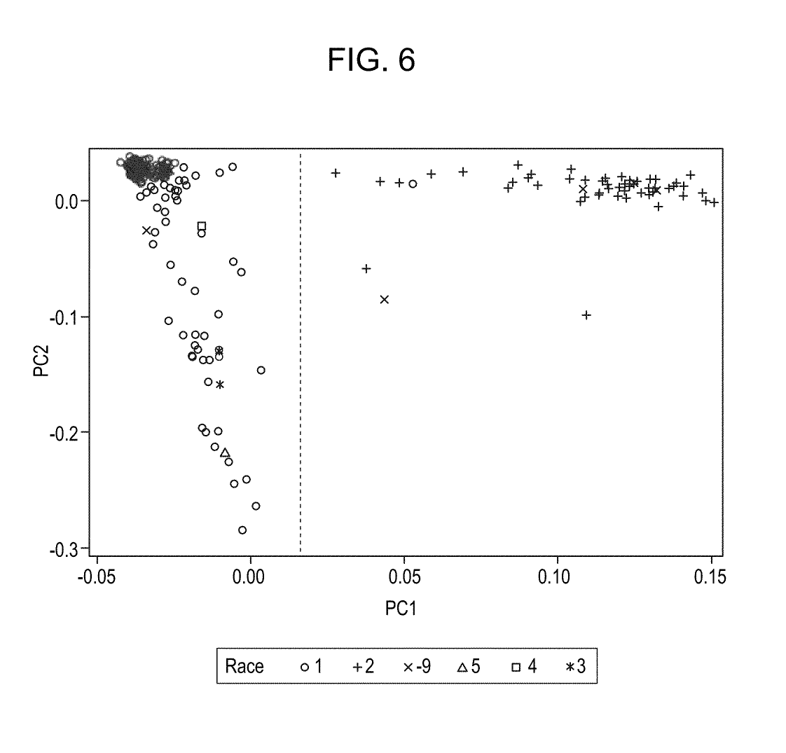

FIG. 6 depicts, in accordance with various embodiments of the invention, Principle Component Analysis used to confirm self-reported race. of is Caucasian American, +2 is African American, x-9 connotes missing data on 9 subjects, and other symbol numbers (*3, □4, and Δ5) represent the subjects that were genetically determined by PCA incongruent to the self-reported ethnicity and therefore were removed from analysis.

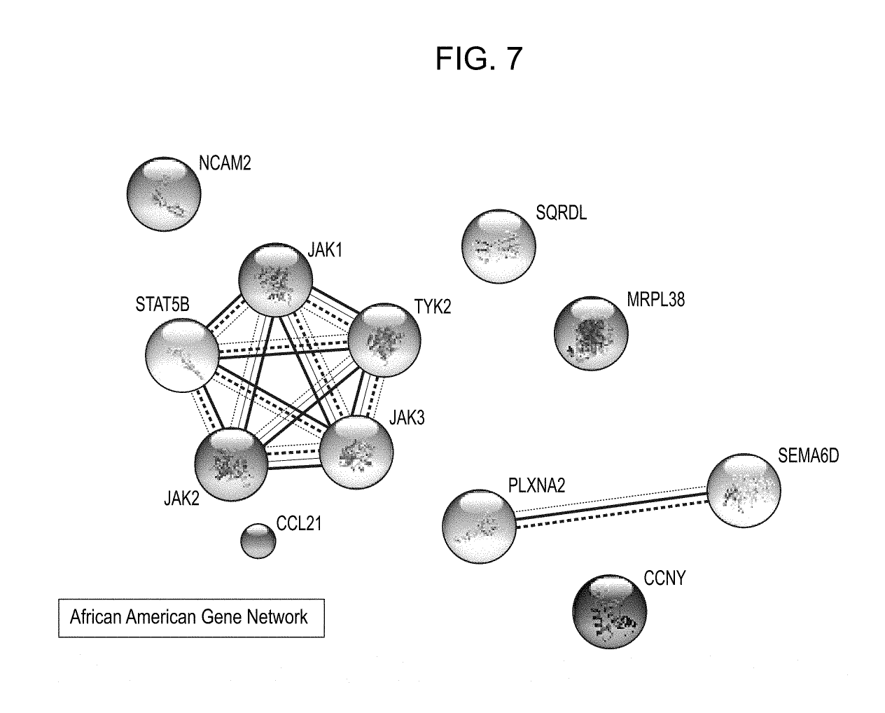

FIG. 7 depicts in accordance with various embodiments of the invention, ndependent protein associations network by racial groups

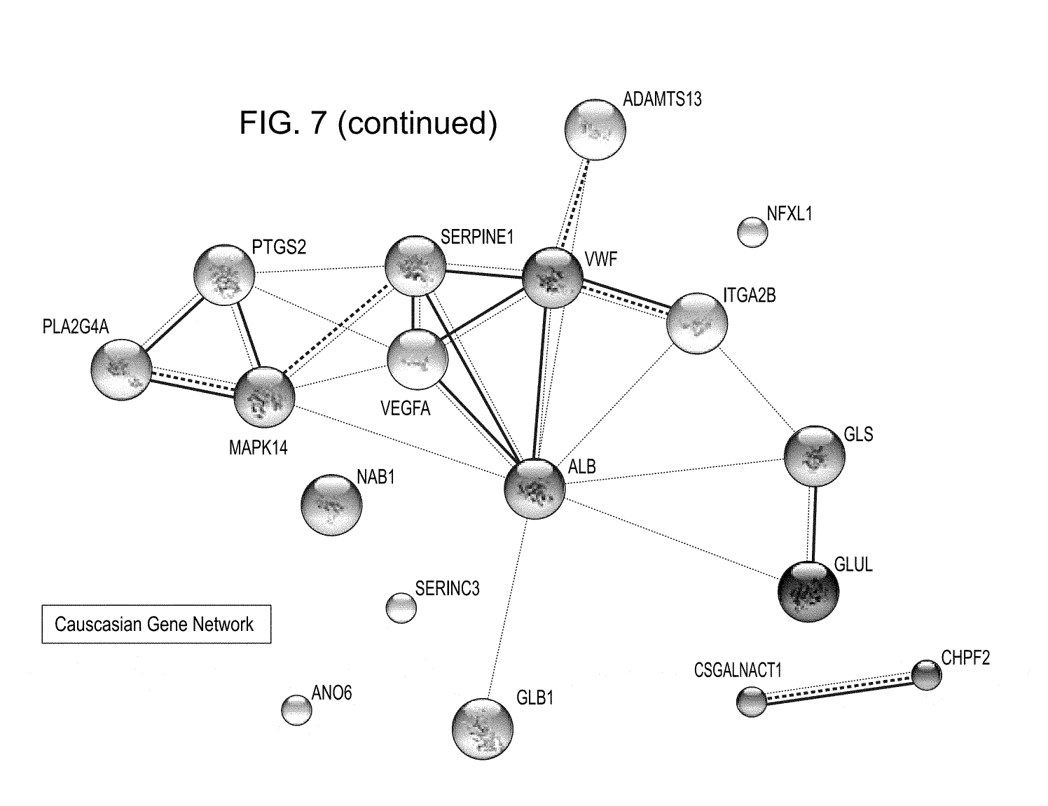

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul, 6(7):511-9; Queen and Selick, Humanized immunoglobulins, U. S. Pat. No. 5,585,089 (1996 Dec); and Riechmann et al., Reshaping human antibodies for therapy, Nature 1988 Mar 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of a cardiovascular condition, delay or slowing of a cardiovascular condition, and amelioration or palliation of symptoms associated with a cardiovascular condition.

“Disorders”, “diseases”, “conditions” and “disease conditions,” as used herein may include, but are in no way limited to any form of cardiovascular conditions, disorders or diseases. Examples of such conditions include but are not limited to end-stage heart failure and severe coronary artery diseases.

The term “sample” or “biological sample” as used herein denotes a sample taken or isolated from a biological organism, e.g., a blood sample from a subject. Exemplary biological samples include, but are not limited to, cheek swab; mucus; whole blood; blood; serum; plasma; urine; saliva; semen; lymph; fecal extract; sputum; other body fluid or biofluid; cell sample; and tissue sample etc. The term also includes a mixture of the above-mentioned samples. The term “sample” also includes untreated or pretreated (or pre-processed) biological samples. In some embodiments, a sample can comprise one or more cells from the subject.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., end-stage heart failure) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

As used herein, the terms “categorizing”, “classifying”, “stratifying”, “subtyping”, and “subgrouping” are interchangeable. As used herein, the terms “category”, “class”, “strata”, “subtype”, and “subgroup” are interchangeable.

“Poor Prognosis” means that the prospect of survival and recovery after a heart transplant in the subject is less than the median survival rate of heart transplants in a sample population. In an embodiment, a subject with poor prognosis is at an increased risk of transplant rejection. In one embodiment, the subject is of black ancestry and the sample population is of black ancestry. In another embodiment, the subject is of black ancestry and the sample population is if Caucasian ancestry. In a further embodiment, the subject is of Caucasian ancestry and the sample population is of black ancestry. In another embodiment, the subject is of Caucasian ancestry and the sample population is Caucasian ancestry. In some embodiments, a poor prognosis is indicative of poor clinical outcome, wherein a poor clinical outcome comprises lower survival rates compared to the mean or median survival rate of reported heart transplant population. In one embodiment, the sample heart transplant population is of the same ancestry as the subject. In another embodiment, the sample heart transplant population is the reported heart transplant population of all ancestries.

“Good Prognosis” means that the prospect of survival and recovery after a heart transplant in the subject is the same as or higher than the median survival rate of heart transplants in a sample population. In an embodiment, a subject with good prognosis is at a decreased risk of transplant rejection. In one embodiment, the subject is of black ancestry and the sample population is of black ancestry. In another embodiment, the subject is of black ancestry and the sample population is if Caucasian ancestry. In a further embodiment, the subject is of Caucasian ancestry and the sample population is of black ancestry. In another embodiment, the subject is of Caucasian ancestry and the sample population is Caucasian ancestry. In some embodiments, a good prognosis is indicative of a good clinical outcome, wherein a good clinical outcome comprises same as or higher survival rates compared to the mean or median survival rate of reported heart transplant population. In one embodiment, the sample heart transplant population is of the same ancestry as the subject. In another embodiment, the sample heart transplant population is the reported heart transplant population of all ancestries.

As used herein, the terms “black”, “of black ethnicity”, “of black race”, “of black ancestry”, “African”, “of African ethnicity”, “of African race” or “of African ancestry” are interchangeable. In the context of the instant application, these ethnic/racial terms adopt their scientific meaning as understood in the biomedical field of population genetics, and refer to those individuals who have origins in any of the original peoples of Sub-Saharan Africa. In the context of the instant application, these ethnic/racial terms are used to describe a subject's biological nature, and should not be confused with their cultural or social meanings as used in non-biomedical fields.

As used herein, the terms “white”, “Caucasian”, “European”, “of European ethnicity”, “of European ancestry” are interchangeable. In the context of the instant application, these ethnic/racial terms adopt their scientific meaning as understood in the biomedical field of population genetics, and refer to those individuals having origins in any of the original peoples of Europe, the Middle East, or North Africa. In the context of the instant application, these ethnic/racial terms are used to describe a subject's biological nature, and should not be confused with their cultural or social meanings as used in non-biomedical fields.

“IL-6 inhibitor” as used herein refers to a therapeutic agent that inhibits IL-6 directly or indirectly (for example, via the IL-6 receptor). Examples of the IL-6 inhibitor include but are not limited to Tocilizumab, Siltuximab and Olokizumab. In an embodiment, the IL-6 inhibitor is Tocilizumab. In some embodiments, the IL-6 inhibitor (for example, Tocilizumab, Siltuximab and/or Olokizumab) is administered intravenously.

“JAK-STAT” inhibitors as used herein refer to agents that inhibit the activity of one or more of the Janus kinase family of enzymes (JAK1, JAK2, JAK3, TYK2), thereby interfering with the JAK-STAT signaling pathway. Examples of JAK-STAT inhibitors include but are not limited to baricitinib, decernotinid, filgotinig, INCB-039110 and tofacitinib.

As described herein, in some embodiments, a patient's ethnicity or race may be self-reported or reported by an investigator (e.g., nurses and physicians), and has been scientifically validated to be accurate for practicing various embodiments of the present invention. In some embodiments, the patient's ethnicity or race may be determined or confirmed with Principal Component Analysis (PCA) and/or mitochondrial DNA (mtDNA) haplotypes. As such, one's African ethnicity or race may be determined via self-reporting, investigator reporting, PCA, mtNDA hyplotypes, or their combinations.

Ethnic/racial disparities after heart transplantation has persisted despite improvement in our knowledge of heart transplant science, immunosuppression options and treatment regimens. Discoveries in immunosuppressive medications have certainly improved heart transplant outcomes over the decades. But to date, nothing has been developed to screen genetic risk in the populations of interest.

Without wishing to be bound by any particular theory, the inventors believe that the basis for increased risk of cardiovascular (CV) death in African American recipients may be due to a higher prevalence of pro-inflammatory polymorphisms in immune response genes that could mediate rejection and might have contributed to injury of the original heart. Our data demonstrated that AfAm had significantly higher frequency of acute rejection episodes at 1 year compared to CaAm heart transplant recipients. Investigations explored the relationship of SNP or DNA-based gene variation in cytokines as a mediator of rejection. A higher distribution of IL-6 GG-174 rs1800797 in the AfAm compared to CaAm renal transplant recipients has been demonstrated. IL-6 is a mediator of acute phase inflammation. Peripheral blood mononuclear cells from AfAm patients responded to stimulation by exhibiting a larger increase in CD80 (B7-1) and CD152 (B7-2) on the antigen presenting cells, which the authors, without wishing to be bound by any particular theory, attributed to higher IL-6 production associated with the GG allele. This immunogenetic risk could contribute to increased inflammatory T-cell responses driving graft rejection. A gene expression panel (Allomap) predicting rejection has been validated and used in practice and its predictive value for AfAm is emerging (Khush, K, et al, 2015 Gene expression profiling to study racial differences after heart transplant. Journal of Heart Lung Transplantation 34; 970-977); notably, IL-6 is not on the Allomap panel.

The isolation of the genes found in this study can be used for risk stratification of heart transplant patients and population health in predicting outcomes. The advantages of the invention described herein include identifying patients at risk for a poor outcome prior to heart transplant and developing individualized treatment plans that are a precision medicine approach to transplantation. Further, the methods described herein may be used to screen patients with heart disease at diagnosis and tailor their treatment to prevent late stage heart failure. Lastly, we could use the tool described herein at birth to identify person at risk for heart failure with the intent of impacting populations by delivering preventative interventions that could impact environmental epigenetic factors.

Accordingly, various embodiments of the present invention provide a method of prognosing a heart transplant in a subject desiring determination of heart transplant prognosis. The method may consist of, or may consist essentially of, or may comprise: obtaining a sample from the subject; assaying the sample to detect the risk alleles at one or more SNPs; calculating a genetic risk score (GRS) of the subject based on the detected risk alleles at the one or more SNPs; and prognosing the heart transplant in the subject based on the GRS risk score of the subject. In an embodiment, the subject has not yet undergone heart transplant. In an embodiment, the subject is of black ancestry. In another embodiment, the subject is of white ancestry. In one embodiment, a GRS risk score same as or below median or mean GRS risk score of the sample population of the same ancestry as the subject is indicative of good prognosis. In another embodiment, a GRS risk score above median or mean GRS risk score of the sample population of the same ancestry as the subject is indicative of poor prognosis.

Also provided herein is a method for determining the likelihood of heart transplant rejection in a subject desiring determination of heart transplant rejection. The method may consist of, or may consist essentially of, or may comprise: obtaining a sample from the subject; assaying the sample to detect the risk alleles at one or more SNPs; calculating a genetic risk score (GRS) of the subject based on the detected risk alleles at the one or more SNPs; and determining that the subject has a decreased likelihood of heart transplant rejection if the GRS of the subject is same as or below median or mean GRS of the sample population of the same ancestry as the subject and determining that the subject has an increased likelihood of heart transplant rejection if the GRS of the subject is above the median or mean GRS of the sample population of the same ancestry as the subject. In an embodiment, the subject has not yet undergone heart transplant. In an embodiment, the subject is of black ancestry. In another embodiment, the subject is of white ancestry.

Also provided herein is a method for determining the likelihood of heart transplant rejection in a subject desiring determination of heart transplant rejection. The method may consist of, or may consist essentially of, or may comprise: detecting the genetic risk score (GRS) of the subject based on the detected risk alleles at the one or more SNPs; and determining that the subject has an decreased likelihood of heart transplant rejection if the GRS of the subject is same as or below median or mean GRS of the sample population of the same ancestry as the subject and determining that the subject has an increased likelihood of heart transplant rejection if the GRS of the subject is above the median or mean GRS of the sample population of the same ancestry as the subject. In an embodiment, the subject has not yet undergone heart transplant. In an embodiment, the subject is of black ancestry. In another embodiment, the subject is of white ancestry.

Various embodiments of the present invention provide a method of classifying a subject with a cardiovascular condition. The method may consist of, or may consist essentially of, or may comprise: obtaining a sample from the subject; assaying the sample to detect the risk alleles at one or more SNPs; calculating a genetic risk score (GRS) of the subject based on the detected risk alleles at the one or more SNPs; and classifying the subject into a group based on the GRS of the subject. In some embodiments, the GRS of the subject is below the median or mean GRS of the population of the same ancestry as the subject, and the subject is classified into a low risk (for cardiovascular diseases) group. In other embodiments, the GRS of the subject is above the median or mean GRS of the population of the same ancestry as the subject, and the subject is classified into a high risk (for cardiovascular diseases) group. In various embodiments, the subject's low or high risk is in comparison to the population of the same ancestry as the subject.

In various embodiments, the methods provided herein further comprises instructing, directing, or informing the subject classified in the low risk GRS group as suitable for a heart transplant to proceed with listing for heart transplant. In some embodiments, the method further comprises evaluation, determination and selection of suitable donor and conducting a heart transplant.

### Poor Prognosis

In an embodiment, the GRS of the subject is above the median or mean GRS of the sample population of the same ancestry as the subject and is indicative of poor prognosis after heart transplant. In an embodiment, if the subject is of black ancestry and the prognosis is poor, the subject is recommended for a heart transplant and is administered an effective amount of an IL-6 inhibitor, a JAK-STAT inhibitor or combinations thereof, wherein the inhibitors are administered before the heart transplant. In an embodiment, if the subject is of black ancestry and the prognosis is poor, the subject is recommended for a heart transplant and is administered an effective amount of an IL-6 inhibitor, a JAK-STAT inhibitor or combinations thereof, wherein the inhibitors are administered during transplant. In an embodiment, if the subject is of black ancestry and the prognosis is poor, the subject is recommended for a heart transplant and is administered an effective amount of an IL-6 inhibitor, a JAK-STAT inhibitor or combinations thereof, wherein the inhibitors are administered after the heart transplant. In an embodiment, if the prognosis is poor, the subject is recommended for a heart transplant and is administered an effective amount of an IL-6 inhibitor, a JAK-STAT inhibitor or combinations thereof, wherein the inhibitors are administered before, during and/or after the heart transplant.

In an embodiment, the subject identified as having increased likelihood of poor prognosis is of black ancestry and expresses any one, two, three, four, five, six, seven, eight or more of risk alleles selected from T in SNP rs12030062 (SEQ ID NO: 1) at position 501, risk allele C in SNP rs2727438 (SEQ ID NO: 2) at position 501, risk allele C in SNP rs73266737 (SEQ ID NO: 3) at position 251, risk allele A in SNP rs8032616 (SEQ ID NO: 4) at position 501, risk allele G in SNP rs10519060 (SEQ ID NO: 5) at position 501, risk allele T in SNP rs3785437 (SEQ ID NO: 6) at position 501, risk allele T in SNP rs7221109 (SEQ ID NO: 7) at position 501, risk allele T in SNP rs62076937 (SEQ ID NO: 8) at position 251, risk allele A in SNP rs2826929 (SEQ ID NO: 9) at position 501 or combinations thereof. In some embodiments, if the subject is homozygous for the risk allele, the prognosis is worse compared to if the subject is heterozygous for the risk allele.

### Good Prognosis

In various embodiments, the GRS of the subject is the same as or above the median or mean GRS of the sample population of the same ancestry as the subject and the prognosis after heart transplant is good. In an embodiment, if the prognosis is good, the subject is recommended for a heart transplant and is not administered an IL-6 inhibitor and/or a JAK-STAT inhibitor. In an embodiment, if the prognosis is good, the subject is recommended for a heart transplant and is optionally administered an IL-6 inhibitor and/or a JAK-STAT inhibitor before, during and/or after the heart transplant at for example, a lower dosage and/or frequency compared to subjects with poor prognosis.

In an embodiment, the subject identified as having increased likelihood of good prognosis is of black ancestry and expresses any one, two, three, four, five, six, seven, eight or more of protective alleles selected from C in SNP rs12030062 (SEQ ID NO: 1) at position 501, protective allele A in SNP rs2727438 (SEQ ID NO: 2) at position 501, protective allele T in SNP rs73266737 (SEQ ID NO: 3) at position 251, protective allele C in SNP rs8032616 (SEQ ID NO: 4) at position 501, protective allele A in SNP rs10519060 (SEQ ID NO: 5) at position 501, protective allele G in SNP rs3785437 (SEQ ID NO: 6) at position 501, protective allele C in SNP rs7221109 (SEQ ID NO: 7) at position 501, protective allele C in SNP rs62076937 (SEQ ID NO: 8) at position 251, protective allele Gin SNP rs2826929 (SEQ ID NO: 9) at position 501 or combinations thereof. In some embodiments, if the subject is homozygous for the protective allele, the prognosis is better compared to if the subject is heterozygous for the protective allele.

### Treatment Methods

Various embodiments of the present invention provide a method of treating a cardiovascular condition in a subject. The method may consist of, or may consist essentially of, or may comprise: providing a donor heart; and conducting a heart transplant on the subject using the donor heart, thereby treating the cardiovascular condition in the subject. In one embodiment, the subject is classified into a low risk (good prognosis) group using a method described herein. In another embodiment, the subject is classified into a high risk (poor prognosis) group and is administered an effective amount of an IL-6 inhibitor, a JAK-STAT inhibitor or combinations thereof, wherein the inhibitors are administered before, during and/or after the heart transplant, as described herein. In an embodiment, the subject is of black ancestry. In an embodiment, the subject is of Caucasian ancestry.

Various embodiments of the present invention provide a method of treating a cardiovascular condition in a subject. The method may consist of, or may consist essentially of, or may comprise: providing a donor heart; and conducting a heart transplant on the subject using the donor heart, thereby treating the cardiovascular condition in the subject, wherein the subject is identified as suitable for a heart transplant using a method described herein.

Various embodiments of the present invention provide a method of treating a cardiovascular condition in a subject. The method may consist of, or may consist essentially of, or may comprise: providing a donor heart; and conducting a heart transplant on the subject using the donor heart, thereby treating the cardiovascular condition in the subject, wherein the subject is directed to receive a heart transplant using a method described herein.

Various embodiments of the present invention provide a method of treating a cardiovascular condition in a subject. The method may consist of, or may consist essentially of, or may comprise: obtaining a sample from the subject; assaying the sample to detect the risk alleles at one or more SNPs; calculating a genetic risk score (GRS) of the subject based on the detected risk alleles at the one or more SNPs; determining that the GRS of the subject is below the median or mean GRS of the population of the same ancestry as the subject; and conducting a heart transplant on the subject, thereby treating the cardiovascular condition in the subject.

Various embodiments of the present invention provide a method of treating a cardiovascular condition in a subject. The method may consist of, or may consist essentially of, or may comprise: obtaining a sample from the subject; assaying the sample to detect the risk alleles at one or more SNPs; calculating a genetic risk score (GRS) of the subject based on the detected risk alleles at the one or more SNPs; determining that the GRS of the subject is above the median or mean GRS of the population of the same ancestry as the subject; and conducting a heart transplant in the subject and administering to the subject an effective amount of an IL-6 inhibitor, a JAK-STAT inhibitor or combinations thereof, wherein the inhibitors are administered before, during and/or after the heart transplant, thereby treating the cardiovascular condition in the subject.

In various embodiments of the methods described herein, the IL-6 inhibitor directly or indirectly inhibits IL-6. In some embodiments of the methods described herein, the IL-6 inhibitor directly inhibits IL-6 and is selected from the group consisting of a small molecule, a peptide, an antibody or a fragment thereof that specifically binds IL-6 or IL-6R and a nucleic acid molecule. In one embodiment of the methods described herein, the IL-6 inhibitor indirectly inhibits IL-6 via IL-6 receptor (IL-6R) wherein inhibitor of IL-6R is selected from the group consisting of a small molecule, a peptide, an antibody or a fragment thereof and a nucleic acid molecule. In some embodiments of the methods described herein, the nucleic acid molecule is a siRNA molecule specific for IL-6 or IL-6R. In some embodiments, the inhibitor is a bispecific molecule that specifically binds IL-6 and IL6R, so as to inhibit IL-6. In some embodiments of the methods described herein, the antibody is selected from the group consisting of monoclonal antibody or fragment thereof, a polyclonal antibody or a fragment thereof, chimeric antibodies, humanized antibodies, human antibodies, and a single chain antibody. In an embodiment of the methods described herein, the inhibitor is Tocilizumab, which is an anti-IL-6R antibody. In an embodiment, the IL-6 inhibitor is Siltuximab. In another embodiment, the IL-6 inhibitor is Olokizumab. In some embodiments, the IL-6 inhibitor is any one or more of Tocilizumab, Siltuximab and Olokizumab.

In various embodiments of the methods described herein, the JAK-STAT inhibitor directly or indirectly inhibits the JAK-STAT pathway. In some embodiments of the methods described herein, the JAK-STAT inhibitor directly inhibits JAK kinase and is selected from the group consisting of a small molecule, a peptide, an antibody or a fragment thereof that specifically binds JAK kinase and a nucleic acid molecule. In some embodiments of the methods described herein, the nucleic acid molecule is a siRNA molecule specific for the JAK kinases. In some embodiments of the methods described herein, the antibody is selected from the group consisting of monoclonal antibody or fragment thereof, a polyclonal antibody or a fragment thereof, chimeric antibodies, humanized antibodies, human antibodies, and a single chain antibody. In an embodiment of the methods described herein, the JAK-STAT inhibitor is any one or more of baricitinib, decernotinib, filgotinib, INCB-039110 and tofacitinib.

In some embodiments of the invention, the effective amounts of the IL-6 inhibitor (for example, Tocilizumab, Siltuximab and Olokizumab) and/or JAK-STAT inhibitors (for example, baricitinib, decernotinib, filgotinib, INCB-039110 and tofacitinib) can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In further embodiments of the invention, the effective amount of IL-6 inhibitor (for example, Tocilizumab, Siltuximab and Olokizumab) and/or JAK-STAT inhibitors (for example, baricitinib, decernotinib, filgotinib, INCB-039110 and tofacitinib) for use with the claimed methods may be in the range of 1-5 mg/kg, 5-10 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg or 900-1000 mg/kg.

In additional embodiments, the effective amount of IL-6 inhibitor (for example, Tocilizumab, Siltuximab and Olokizumab) and/or JAK-STAT inhibitors (for example, baricitinib, decernotinib, filgotinib, INCB-039110 and tofacitinib) is about 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-11 mg/kg, 11-12 mg/kg, 12-13 mg/kg, 13-15 mg, 15-20 mg/kg or 20-25 mg/kg.

In one embodiment, the IL-6 inhibitor is Tocilizumab and the dosage is 8 mg/kg. In another embodiment, the IL-6 inhibitor is Siltuximab and the dosage is 12 mg/kg. In a further embodiment, the IL-6 inhibitor is Olokizumab and the dosage is 8 mg/kg.

In one embodiment, the IL-6 inhibitor is administered intravenously. The optimum dosage, regimen, mode of administration and duration of administration will be apparent to a person of skill in the art.

### Biological Samples

In various embodiments, the sample is cheek swab; mucus; whole blood; blood; serum; plasma; urine; saliva; semen; lymph; fecal extract; sputum; other body fluid or biofluid; cell sample; or tissue sample; or a combination thereof. In various embodiments, the sample comprises a nucleic acid from the individual. In some embodiments, the nucleic acid comprises genomic DNA, or mitochondrial DNA, or both. In various embodiments, the sample is a body fluid. In some embodiments, the body fluid is whole blood, plasma, saliva, mucus, or cheek swab. In various embodiments, the sample is a cell or tissue. In some embodiments, the cell is a blood cell. In some embodiments, the cell is a blood cell line (e.g., a lymphoblastoid cell line) obtained from the subject and transformed with an Epstein Barr virus.

### Subjects

In various embodiments, the subject is a human. In some embodiments, the subject is a child. In some embodiments, the subject is a teenager. In other embodiments, the subject is an adult. In various embodiments, the subject is of black ancestry and has a cardiovascular condition. In various embodiments, the subject is of black ancestry and has a cardiovascular condition. In various embodiments, the cardiovascular condition is end-stage heart failure caused by a severe coronary artery, idiopathic or congenital disease. In various embodiments, the subject is waiting for a heart transplant or has received a heart transplant.

### Genetic Risk Score and Diagnostic SNPs

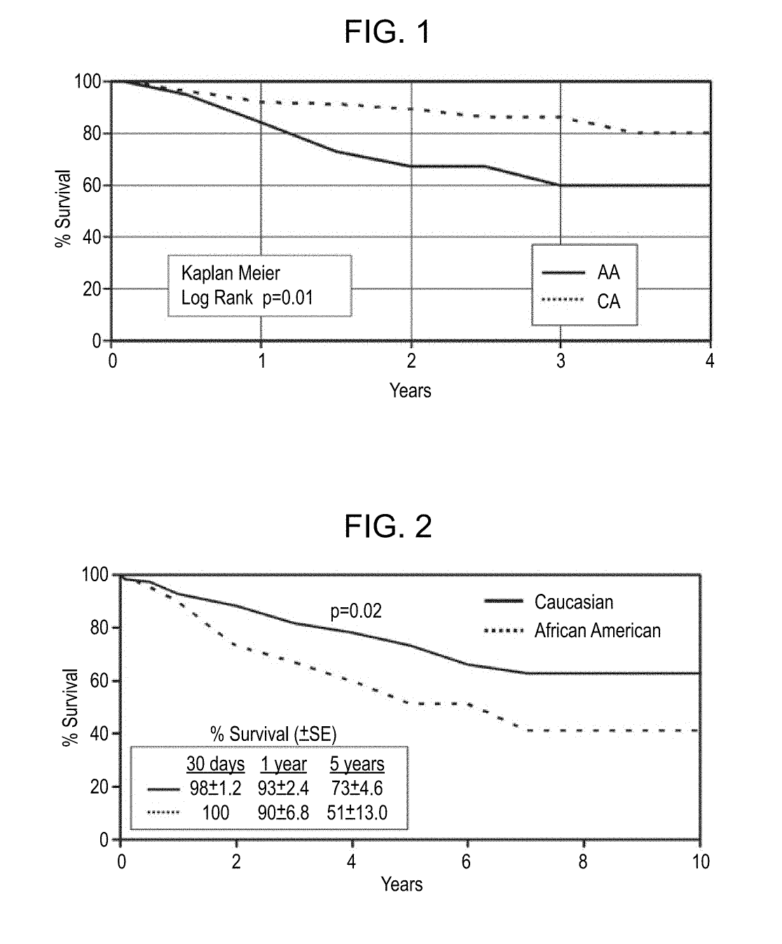

Activation of the inflammatory SNPs which are mutually exclusive between the African American and Caucasian American heart transplant recipients, is activated through two separate gene networks. For the African American group, the inflammatory activation is initiated by intracellular mechanism through the JAK-STAT signaling pathway. In contrast, inflammation in the Caucasian American group is initiated through extracellular mechanisms likely acute phase reactive genes (FIG. 1).

The sample was reclassified into two ethnic groups based upon Principal Component analysis [Salas, A., et al., Charting the ancestry of African Americans. Am J Hum Genet, 2005. 77(4): p. 676-80]. All SNPs with a minor allele frequency (MAF)>1% and missing rate of <5% (and Hardy-Weinberg equilibrium (HWE) p=0.001) were included (102,647) in the analysis. Genotype clusters were manually reviewed to ensure correct allele calling. (Tables 1 and 2). Within each ethnic group, death is determined by Kaplan Meier survival analysis. All SNPs with p-values less than 10⁻⁴ were selected and used to determine the risk allele with coding of homozygote=2, heterozygote=1 and non-risk allele=0. The genetic risk score (GRS) was calculated by summing the risk alleles across the loci for each study participant within each ethnic group. A group median score was determined and subjects were categorized as low risk or high risk relative to the group median. GRS in AfAm and CaAm Htx were determined independently. We further explored the clinical context of these two heart transplant patient groups on select clinical variables of interest that can influence heart transplant outcomes. Significant differences between ethnic groups were identified for recipient age, donor age, gender and disease type (Table 3).

In various embodiments, the GRS is the total number of the detected risk alleles at the one or more SNPs. In some embodiments, the GRS is the total number of the detected risk alleles at all the SNPs listed in Table 1. In other embodiments, the GRS is the total number of the detected risk alleles at all the SNPs listed in Table 2. In various embodiments, the one or more SNPs comprise one, two, three, four, five, six, or more, or all of: rs12030062 (SEQ ID NO: 1), rs2727438 (SEQ ID NO: 2), rs73266737 (SEQ ID NO: 3), rs8032616 (SEQ ID NO: 4), rs10519060 (SEQ ID NO: 5), rs3785437 (SEQ ID NO: 6), rs7221109 (SEQ ID NO: 7), rs62076937(SEQ ID NO: 8), rs2826929 (SEQ ID NO: 9), rs6690278 (SEQ ID NO: 10), rs2355570 (SEQ ID NO: 11), rs115230839 (SEQ ID NO: 12), rs17050452 (SEQ ID NO: 13), rs7688988 (SEQ ID NO: 14), rs80165265 (SEQ ID NO: 15), rs1991764 (SEQ ID NO: 16), rs4922070 (SEQ ID NO: 17), rs7957672 (SEQ ID NO: 18), rs2544081 (SEQ ID NO: 19), rs6564724 (SEQ ID NO: 20), and rs111315210 (SEQ ID NO: 21).

In some embodiments, the subject is of African ancestry. In various embodiments, the one or more SNPs comprise one, two, three, four, five, six, or more, or all of: rs12030062 (SEQ ID NO: 1), rs2727438 (SEQ ID NO: 2), rs73266737 (SEQ ID NO: 3), rs8032616 (SEQ ID NO: 4), rs10519060 (SEQ ID NO: 5), rs3785437 (SEQ ID NO: 6), rs7221109 (SEQ ID NO: 7), rs62076937 (SEQ ID NO: 8), and rs2826929 (SEQ ID NO: 9). In certain embodiments, the GRS is the total number of the detected risk alleles at all of: rs12030062 (SEQ ID NO: 1), rs2727438 (SEQ ID NO: 2), rs73266737 (SEQ ID NO: 3), rs8032616 (SEQ ID NO: 4), rs10519060 (SEQ ID NO: 5), rs3785437 (SEQ ID NO: 6), rs7221109 (SEQ ID NO: 7), rs62076937 (SEQ ID NO: 8), and rs2826929 (SEQ ID NO: 9). In various embodiments, the subject's GRS is compared to the median or mean GRS of the population of African ancestry. In some embodiments, the GRS of the African subject is above the median or mean GRS of the population of African ancestry, and the African subject is classified into a high risk group and/or is identified as suitable for heart transplant and is administered an effective amount of an IL-6 inhibitor, a JAK-STAT inhibitor or combinations thereof, wherein the inhibitors are administered before, during and/or after the heart transplant. In some embodiments, the GRS of the African subject is same as or below the median or mean GRS of the population of African ancestry, and the African subject is classified into a low risk group, and/or identified as suitable for a heart transplant, and/or directed to receive a heart transplant, and/or prognosed with a good clinical outcome for a heart transplant, and/or treated with a heart transplant.

In an embodiment, the subject identified as having increased likelihood of poor prognosis is of black ancestry and expresses any one, two, three, four, five, six, seven, eight or more of risk alleles selected from T in SNP rs12030062 (SEQ ID NO: 1) at position 501, risk allele C in SNP rs2727438 (SEQ ID NO: 2) at position 501, risk allele C in SNP rs73266737 (SEQ ID NO: 3) at position 251, risk allele A in SNP rs8032616 (SEQ ID NO: 4) at position 501, risk allele G in SNP rs10519060 (SEQ ID NO: 5) at position 501, risk allele T in SNP rs3785437 (SEQ ID NO: 6) at position 501, risk allele T in SNP rs7221109 (SEQ ID NO: 7) at position 501, risk allele T in SNP rs62076937 (SEQ ID NO: 8) at position 251, risk allele A in SNP rs2826929 (SEQ ID NO: 9) at position 501 or combinations thereof. In some embodiments, if the subject is homozygous for the risk allele, the prognosis is worse compared to if the subject is heterozygous for the risk allele.

In an embodiment, the subject identified as having increased likelihood of good prognosis is of black ancestry and expresses any one, two, three, four, five, six, seven, eight or more of protective alleles selected from C in SNP rs12030062 (SEQ ID NO: 1) at position 501, protective allele A in SNP rs2727438 (SEQ ID NO: 2) at position 501, protective allele T in SNP rs73266737 (SEQ ID NO: 3) at position 251, protective allele C in SNP rs8032616 (SEQ ID NO: 4) at position 501, protective allele A in SNP rs10519060 (SEQ ID NO: 5) at position 501, protective allele G in SNP rs3785437 (SEQ ID NO: 6) at position 501, protective allele C in SNP rs7221109 (SEQ ID NO: 7) at position 501, protective allele C in SNP rs62076937 (SEQ ID NO: 8) at position 251, protective allele Gin SNP rs2826929 (SEQ ID NO: 9) at position 501 or combinations thereof. In some embodiments, if the subject is homozygous for the protective allele, the prognosis is better compared to if the subject is heterozygous for the protective allele.

In other embodiments, the subject is of European ancestry. In various embodiments, the one or more SNPs comprise one, two, three, four, five, six, or more, or all of: rs6690278 (SEQ ID NO: 10), rs2355570 (SEQ ID NO: 11), rs115230839 (SEQ ID NO: 12), rs17050452 (SEQ ID NO: 13), rs7688988 (SEQ ID NO: 14), rs80165265 (SEQ ID NO: 15), rs1991764 (SEQ ID NO: 16), rs4922070 (SEQ ID NO: 17), rs7957672 (SEQ ID NO: 18), rs2544081 (SEQ ID NO: 19), rs6564724 (SEQ ID NO: 20), and rs111315210 (SEQ ID NO: 21). In certain embodiments, the GRS is the total number of the detected risk alleles at all of: rs6690278 (SEQ ID NO: 10), rs2355570 (SEQ ID NO: 11), rs115230839 (SEQ ID NO: 12), rs17050452 (SEQ ID NO: 13), rs7688988 (SEQ ID NO: 14), rs80165265 (SEQ ID NO: 15), rs1991764 (SEQ ID NO: 16), rs4922070 (SEQ ID NO: 17), rs7957672 (SEQ ID NO: 18), rs2544081 (SEQ ID NO: 19), rs6564724 (SEQ ID NO: 20), and rs111315210 (SEQ ID NO: 21). In various embodiments, the subject's GRS is compared to the median or mean GRS of the population of European ancestry. In some embodiments, the GRS of the European subject is above the median or mean GRS of the population of European ancestry, and the European subject is classified into a high risk group, and/or identified as suitable for a heart transplant but requiring personalized management after heart transplant. In some embodiments, the method further comprises not conducting a heart transplant on subjects who are deemed clinically ineligible by a medical team. In some embodiments, the GRS of the European subject is not above the median or mean GRS of the population of European ancestry, and the European subject is classified into a low risk group, and/or identified as suitable for a heart transplant, and/or directed to receive a heart transplant, and/or prognosed with a good clinical outcome for a heart transplant, and/or treated with a heart transplant.

### Detection Methods

In various embodiments, a SNP's alleles are detected by: contacting the sample with detection agents that specifically bind to the SNP's alleles; and detecting the binding levels between the detection agents and the SNP's alleles. Alleles can be detected by genotyping assays, PCR, Reverse transcription PCR, real-time PCR, microarray, DNA sequencing, and RNA sequencing techniques.

In various embodiments, the detection agents are oligonucleotide probes, nucleic acids, DNAs, RNAs, aptamers, peptides, proteins, antibodies, avimers, or small molecules, or a combination thereof. In some embodiments, the detection agents are allele-specific oligonucleotide probes targeting the SNP's alleles. In various embodiments, a SNP's alleles are detected by using a microarray. In some embodiments, the microarray is an oligonucleotide microarray, DNA microarray, cDNA microarrays, RNA microarray, peptide microarray, protein microarray, or antibody microarray, or a combination thereof.

In various embodiments, assaying the sample to detect a SNP's alleles comprises: contacting the sample with one or more allele-specific oligonucleotide probes targeting the SNP's alleles; generating double-stranded hybridization complex through allele-specific binding between the SNP's alleles and said allele-specific oligonucleotide probes; and detecting the double-stranded hybridization complex newly generated through allele-specific binding between the SNP's alleles and said allele-specific oligonucleotide probes. In some embodiments, the method further comprises conducting PCR amplification of the double-stranded hybridization complex.

In accordance with the present invention, said allele-specific oligonucleotide probes may comprise about 10-15, 15-20, 20-25, 25-30, 30-35, 35-40, 40-45, or 45-50 nucleotides; they are either identical or complementary to a sequence segment encompassing the polymorphic position of a SNP as disclosed herein; and they are specific to one or the other allele at the polymorphic position. For a non-limiting example, rs2727438 (SEQ ID NO: 2) has either A or C allele at its polymorphic position (e.g., “M” at nucleotide 501 of the following exemplar sequence).

In various embodiments, allele-specific oligonucleotides specific to the polymorphic site within each SNP comprise nucleotides specific to the polymorphic site in the SNP. For example, an allele-specific oligonucleotide probe for the A allele at rs2727438 (SEQ ID NO: 2) may comprise, for a non-limiting example, 21 nucleotides; and these 21 nucleotides are either identical or complementary to the sequence segment 481-501, 482-502, 483-503, 484-504, 485-505, 486-506, 487-507, 488-508, 489-509, 490-511, 491-511, 492-512, 493-513, 494-514, 495-515, 496-516, 497-517, 498-518, 499-519, 500-520, or 501-521 of the above exemplar sequence where nucleotide 501 is set as the A allele. Vice versa, an allele-specific oligonucleotide probe for the C allele at rs2727438 (SEQ ID NO: 2) may comprise, for a non-limiting example, 21 nucleotides; and these 21 nucleotides are either identical or complementary to the sequence segment 481-501, 482-502, 483-503, 484-504, 485-505, 486-506, 487-507, 488-508, 489-509, 490-511, 491-511, 492-512, 493-513, 494-514, 495-515, 496-516, 497-517, 498-518, 499-519, 500-520, or 501-521 of the above exemplar sequence where nucleotide 501 is set as the C allele.

In various embodiments, said allele-specific oligonucleotide probes are labeled with one or more fluorescent dyes, and wherein detecting the double-stranded hybridization complex comprises detecting fluorescence signals from the fluorescent dyes. In some embodiments, said allele-specific oligonucleotide probes are labeled with a reporter dye and a quencher dye. In some embodiments, detecting the double-stranded hybridization complex comprises detecting the electrophoretic mobility of the double-stranded hybridization complex.

A variety of methods can be used to detect the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Detecting the presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a variant allele or haplotype. Allele-specific oligonucleotide hybridization is based on the use of a labeled oligonucleotide probe having a sequence perfectly complementary, for example, to the sequence encompassing a variant allele or haplotype. Under appropriate conditions, the allele-specific 4818-5225-1713.10 065472-000594US00 probe hybridizes to a nucleic acid containing the variant allele or haplotype but does not hybridize to the other alleles or haplotypes, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a variant allele or haplotype by using an allele-specific oligonucleotide primer that is perfectly complementary to the nucleotide sequence of the variant allele or haplotype but which has one or more mismatches as compared to other alleles or haplotypes (Mullis et al., supra, (1994)). One skilled in the art understands that the one or more nucleotide mismatches that distinguish between the variant allele or haplotype and the other alleles or haplotypes are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the variant allele or haplotype and the other alleles at the 3′ end of the primer.

A heteroduplex mobility assay (HMA) is another well-known assay that may be used to detect a variant allele or haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a variant allele or haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a variant allele or haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a variant allele or haplotype are known in the art and useful in the methods of the invention. Other well-known approaches for determining the presence or absence of a variant allele or haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotypes is to be determined, individual alleles or haplotypes can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention may be practiced using one or any combination of the well-known assays described above or another art-recognized genetic assay.

### Selecting Therapies to Reduce Risk of Heart Transplant Rejection

Various embodiments of the methods described herein further comprise selecting a therapy to reduce or inhibit risk of heart transplant in a subject in need thereof. In some embodiments, the method includes obtaining a sample from the subject; assaying the sample to detect the risk alleles at one or more SNPs; calculating a genetic risk score (GRS) of the subject based on the detected risk alleles at the one or more SNPs; prognosing the heart transplant in the subject based on the GRS risk score of the subject; and selecting a therapy. In an embodiment, the subject has not yet undergone heart transplant. In an embodiment, the subject is of black ancestry. In another embodiment, the subject is of white ancestry. In one embodiment, a GRS risk score same as or below median or mean GRS risk score of the sample population of the same ancestry as the subject is indicative of good prognosis. In another embodiment, a GRS risk score above median or mean GRS risk score of the sample population of the same ancestry as the subject is indicative of poor prognosis. In an embodiment, if the subject has an increased likelihood of good prognosis, the selected therapy is a heart transplant. In an embodiment, if the subject has an increased likelihood of poor prognosis, the selected therapy is a heart transplant and the subject is administered an effective amount of an IL-6 inhibitor, a JAK-STAT inhibitor or combinations thereof, wherein the inhibitors are administered before, during and/or after the heart transplant.

### Compositions

Various embodiments of the present invention provide a composition. This composition may be used for classifying a subject with a cardiovascular condition, and/or identifying the subject as suitable for heart transplant and not requiring an IL-6 and/or JAK-STAT inhibitorsand/or identifying the subject suitable of transplant and requiring an IL-6 and/or JAK-STAT inhibitors before, during and/or after heart transplant. In various embodiments, the composition comprises one or more detection agents that specifically bind to one or more SNPs' alleles. In various embodiments, the composition further comprises a sample from the subject.

### Kits

Various embodiments of the present invention also provide a kit. The kit may consist of or may consist essentially of or may comprise: a composition as described herein, and instructions for using the composition for classifying a subject with a cardiovascular condition, and/or identifying the subject as suitable for heart transplant and not requiring an IL-6 and/or JAK-STAT inhibitors and/or identifying the subject suitable of transplant and requiring an IL-6 and/or JAK-STAT inhibitors before, during and/or after heart transplant. In various embodiments, the kit further comprises a sample from the subject.

Various embodiments of the present invention also provide a kit. The kit may consist of or may consist essentially of or may comprise: one or more detection agents that specifically bind to one or more SNP's alleles, and instructions for using the composition for classifying a subject with a cardiovascular condition, and/or identifying the subject as suitable for heart transplant and not requiring an IL-6 and/or JAK-STAT inhibitors and/or identifying the subject suitable of transplant and requiring an IL-6 and/or JAK-STAT inhibitors before, during and/or after heart transplant. In various embodiments, the kit further comprises a sample from the subject.

In various embodiments, the subject desires a classification into a low or high risk group for a heart transplant, and/or identification as suitable, requiring individualize/personal post-transplant management, or not suitable for a heart transplant, and/or a prognosis of the clinical outcome of a heart transplant.

The kit is an assemblage of materials or components, including at least one of the inventive elements or modules. Thus, in some embodiments the kit contains one or more detection agents that specifically bind to one or more SNP's alleles, as described above; and in other embodiments the kit contains a sample obtained from the subject, as described above.

In various embodiments, the one or more detection agents are applied to contact a biological sample obtained from the subject; and the level of binding between the one or more detection agents and the one or more SNP's alleles is detected to calculate GRS. In some embodiments, the one or more detection agents are oligonucleotide probes, nucleic acids, DNAs, RNAs, peptides, proteins, antibodies, aptamers, or small molecules, or a combination thereof. In various embodiments, the level of binding is detected using a microarray. In some embodiments, the microarray is an oligonucleotide microarray, DNA microarray, cDNA microarrays, RNA microarray, peptide microarray, protein microarray, or antibody microarray, or a combination thereof.

The exact nature of the components configured in the inventive kit depends on its intended purpose. Instructions for use may be included in the kit. “Instructions for use” typically include a tangible expression describing the technique to be employed in using the components of the kit to affect a desired outcome. Optionally, the kit also contains other useful components, such as, spray bottles or cans, diluents, buffers, pharmaceutically acceptable carriers, syringes, catheters, applicators (for example, applicators of cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the detection agents can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

### Reference Values

Various methods described herein may compare a subject's GRS to a pre-determined reference GRS value.

In various embodiments, the reference GRS value is the median or mean GRS of the general population of the same ancestry as the subject. For non-limiting examples, if the subject is an African, then the subject's GRS is compared to the median or mean GRS of the general population of African ancestry; or if the subject is a Caucasian, then the subject's GRS is compared to the median or mean GRS of the general population of European ancestry.

In some embodiments, the reference GRS value is the median or mean GRS of the healthy population of the same ancestry as the subject. For non-limiting examples, if the subject is an African, then the subject's GRS is compared to the median or mean GRS of the healthy population of African ancestry; or if the subject is a Caucasian, then the subject's GRS is compared to the median or mean GRS of the healthy population of European ancestry. As used herein, “healthy” means no need of a heart transplant.

In other embodiments, the reference GRS value is the median or mean GRS of the patient population of the same ancestry as the subject. For non-limiting examples, if the subject is an African, then the subject's GRS is compared to the median or mean GRS of the patient population of African ancestry; or if the subject is a Caucasian, then the subject's GRS is compared to the median or mean GRS of the patient population of European ancestry. As used herein, “healthy” means no need of a heart transplant.

Reference values may be obtained by various methods known in the field. For example, one or more samples from one individual may be collected, processed and analyzed to obtain the individual's GRS value (hereinafter “GRS-1”). The same step is used to obtain GRS values in another 10, 100, 200, 300, 400, 500, 600, 700, 800, 900, 1000 or more individuals, that is, “GRS-n” (n is 1, 2, 3, 4, 5, 6, 7, . . . ). Then, the median or mean of GRS-n may be used as the reference GRS value, to which the subject's GRS is compared to.

Various statistical methods, for example, a two-tailed student t-test with unequal variation, may be used to measure the differences between the subject's GRS and a reference GRS value generated by pooling many individuals of the same ancestry as the subject, as described herein. A significant difference may be achieved where the p value is equal to or less than 0.05.

In various embodiments, the subject's GRS as compared to the reference GRS value is higher by at least or about 5, 10, 15, 20, 25, 30, 35, 40, 45, 50, 55, 60, 65, 70, 75, 80, 85, 90, 95, or 100%. In various embodiments, the subject's GRS as compared to the reference GRS value is lower by at least or about 5, 10, 15, 20, 25, 30, 35, 40, 45, 50, 55, 60, 65, 70, 75, 80, 85, 90, 95, or 100%. In various embodiments, the ratio between the subject's GRS and the reference GRS value is at least or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 4:1, 5:1, 6:1, 7:1, 8:1, 9:1 or 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, or 100:1. In various embodiments, the ratio between the reference GRS value and the subject's GRS is at least or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 4:1, 5:1, 6:1, 7:1, 8:1, 9:1 or 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, or 100:1.

Many variations and alternative elements have been disclosed in embodiments of the present invention. Still further variations and alternate elements will be apparent to one of skill in the art. Among these variations, without limitation, are the selection of constituent modules for the inventive methods, compositions, kits, and systems, and the various conditions, diseases, and disorders that may be diagnosed, prognosed or treated therewith. Various embodiments of the invention can specifically include or exclude any of these variations or elements.

Although the open-ended term “comprising,” as a synonym of terms such as including, containing, or having, is used herein to describe and claim the invention, the present invention, or embodiments thereof, may alternatively be described using alternative terms such as “consisting of or “consisting essentially of.”

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The invention will be further explained by the following Examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Pre-Transplant Inflammatory SNP Characterize Genetic Risk of African American and Caucasian Heart Transplant Patients

Solid organ transplantation is often the last treatment option for patients with end stage heart disease unresponsive to medical management. In 2010 within the United States, 1903 heart transplants were performed in Region one (which includes California). There are 3158 patients within Region 5 awaiting heart transplantation (Organ Procurement and Transplant Network OPTN, 2010). This illustrates the great scarcity of donor hearts for all who are eligible and await heart transplant and improved quality of life.

An issue germane to heart transplantation is the national disparity in long term survival when comparing African American to Caucasian American heart transplant recipient outcomes. Differences in late survival rates (AA-62.3% (CI 61.1-66.9) versus EA-73.3% (CI 72.2-74.3) at five years after heart transplantation are significantly worse for African Americans (OPTN, 2010). Concerning is the disparity in survival when comparing African American to Caucasian American heart transplant recipients. Preventing rejection of the transplanted organ is the fundamental reason for the required immunosuppressive regimens patients must take to promote graft/patient survival.

Incidence and severity of rejection is variable among ethnic groups. African Americans have a higher incidence of allograft failure post heart transplant, than patients of other races. Reasons for this increased incidence in allograft rejection are likely to be attributed to both immunologic and non-immunologic factors. However, HLA-DR mismatch is commonly postulated as a culprit and has been suggested as a contributor to decreased survival in both African American heart transplant patients and others. HLA mismatch notwithstanding, allograft rejection is mediated by activation of T cell, B cells and cytokines.

Genetic variation within the immune response, including cytokine genes that medicate rejection, have emerged as a potential contributor to both individual and population differences observed in heart transplant rejection patterns observed after solid organ transplantation

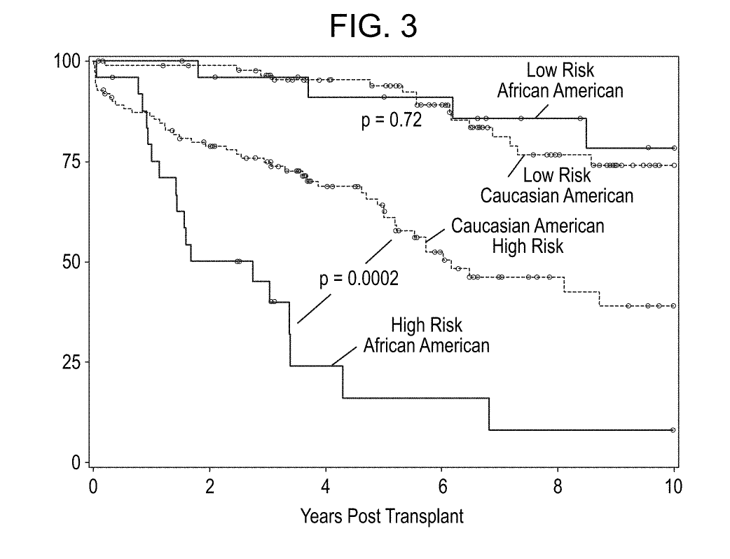

We determined the impact of cytokine gene polymorphism using DNA from lymphocytes obtained during the pre-transplant period (N=20 AfAm and N=114 CaAm) on differential ethnic/racial outcomes after heart transplantation. Using the Qiagen Cytokine Genotyping Tray testing 15 SNPs on five genes IL-6, IFN-γ, TGFB-1, IL-10 and TNF-α, we found three SNPs that were associated with rejection after heart transplantation. Homozygosity for Interleukin-6 (IL-6) GG at rs2430561, tumor necrosis factor-α (TNF-α) GG rs1800629 and interferon-γ (IFN-γ) AA at rs2430561 had a negative impact on survival. Self-reported AfAm heart transplant recipients with the combined IL-6 GG and TNFα GG polymorphisms were found to have worse survival compared to CaAm recipients with the same combined SNP genotypes (FIG. 1). Similar impact upon survival was demonstrated between these groups of interest for subjects found to demonstrate the IFN-γ AA genotype (FIG. 2). The importance of these pro-inflammatory cytokines (IL-6, TNFα, and IFN-γ) is their mediation of acute and antibody-mediated rejection associated with poor survival in AfAm. We investigated other informative SNPs and their relationship to inflammatory processes as well as to inquire whether mtDNA variation might modify risk in these groups.

The current study is a retrospective observational study using a Illumina version 1 Immunochip platform to exploring the ethnic/racial differences and associations between SNPS and potential genetic pathways associated with rejection and survival. The groups of interest are African American and Caucasian American heart transplant recipients.

### Inclusion Criteria

Heart transplant recipients who: 1. Have undergone heart transplantation for the first time; 2. Have available DNA or leukocytes obtained during the pre-transplant evaluation period banked in the participating Center's HLA laboratory; 3. Self-reported ethnicity of African American; 4. Histologically documented acute rejection defined by the International Society of Heart Lung Transplantation (ISHLT) as grade 1a, 1b, 3a, 3b 4a, and 4b; and 5.Are receiving an immunosuppressive regimen to include a calcineurin inhibitor, MMF or Immuran preparation and steroids post heart transplantation.

Banked DNA from the HLA laboratory form African American/Caucasian American heart transplant recipients were used to explore presence of inflammatory SNPs with ethnic/racial groups and compare survival at 10 years between ethnic/racial groups.

### Exclusion Criteria

Heart transplant recipients who: 1. Self-reported ethnicity other than African American or Caucasian American; 2. Undergone a double organ transplant or re-transplant; 3. Do not have banked DNA or leukocytes in the HLA laboratory; and 4. Inadequate clinical or demographic information.

### Patient and Clinical Variables of Interest

1. Age; 2. Gender; 3. Disease type; 4. DM; 5. NYHA Class; 6. CMV status; 7. Donor/recipient HLA DR match (retrospective); 8. PRA (protein reactive antibody); 9. Documentation of all positive acute rejection episodes within the time frame of day of transplant to 10 years post-heart transplantation; 10. Donor age; 11. Donor gender; 12. Ischemic time; 13. Punp Time; 14. Calcineurin dose; 15. Calcineurin Trough level; 16. Steroid dosage; and 17. overall rejection status.

### Study Procedures

Heart Transplant Recipient Recruitment/ Sample Size: A Sample size for 53 African American heart transplant recipients who have available DNA or banks leukocytes is included. A sample size of 204 consecutive DNA specimens obtained from Caucasian American is included.

Specimen Collection: Heart Transplant Recipients DNA. This retrospective study uses deoxyribonucleic acid (DNA) extracted by HLA staff from previously frozen mononuclear leukocytes obtained from both African American and Caucasian American heart transplant recipients obtained during the pre-transplant evaluation period. This material (DNA) is otherwise not needed for clinical care and remains banked as extra sample in the HLA laboratory at Cedars Sinai Medical Center. If DNA has not been extracted from the banked frozen mononuclear leukocytes, it is extracted by the Genetics Core Laboratory.

### Data Accuracy and Protocol

DNA banked in the HLA laboratory were analyzed using the inflammatory platform of Immunochip version 1. In cases where DNA is not available, lymphocytes were transferred to laboratory for DNA extraction. Prior to transfer, the samples are given a code. Data associated with heart transplant subjects in this study are assigned an anonymous code (without unique identifiers e.g., name, social security number, medical record number) to assure correct association of the laboratory samples clinical variables of interest. This code allows both the individual DNA samples and their clinical variables to be directly linked. Without direct linking of the DNA samples with the clinical variables of interest the study cannot be conducted. A second data file is created using a second set of anonymous codes using random assignment for data derived from the DNA samples and the clinical variables of interest. Like the first data file, the second data file does not have unique identifiers. All source documents, for example, preexisting cardiac transplant databases or transplant records, are stored.

### Example 2

### Development of a Genetic Risk Score as Predictor of Ethnic/racial Survival after Heart Transplantation

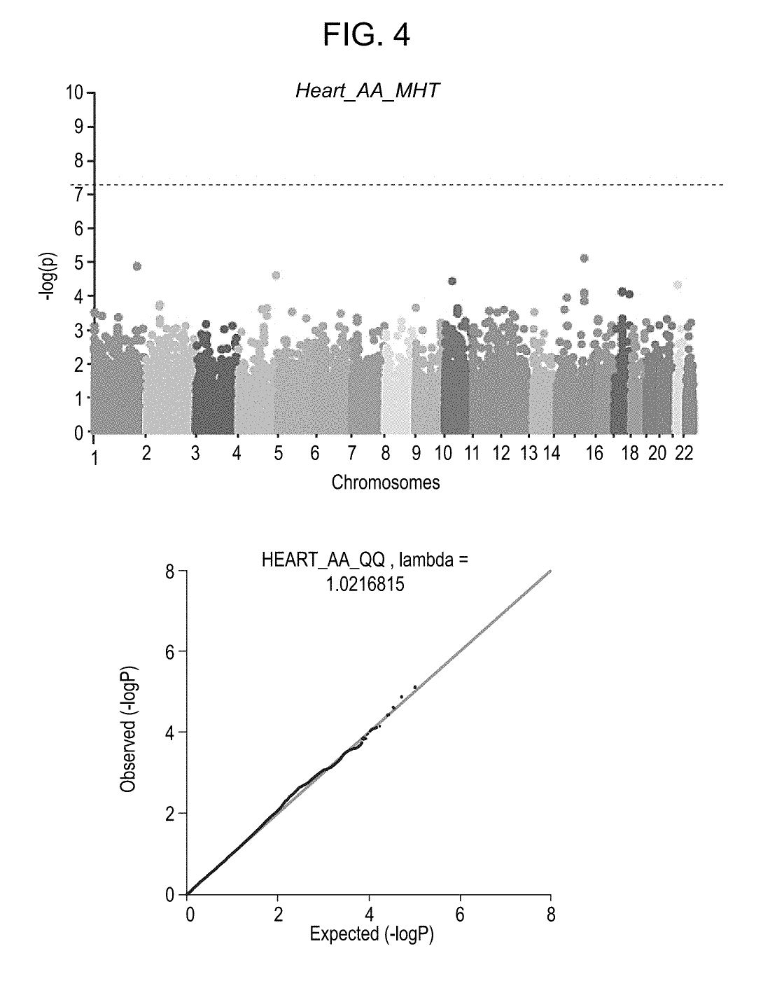

We investigate the underlying reason why African-American heart transplant patients had lower survival rates than Caucasian American patients. While disparities in heart transplantation outcomes between African Americans and Caucasian Americans are known, the role of genetics in predicting survival has not been established. In this study, we uncovered genetic differences among African Americans and Caucasian Americans that are linked to the discrepancy in survival rates between the two ethnic/racial groups. This study has found supporting evidence that the Genetic Risk Score (GRS) predicts worse survival in African American heart transplant recipients at 10 years (FIG. 3).

Heart transplantation, while the only definitive treatment for end-stage heart failure in 2015, continues to demonstrate a disparity in survival outcomes between African Americans and Caucasian American recipients. Without wishing to be bound by any particular theory, we believe there is a link between select single nucleotide polymorphisms (SNP) related to inflammatory processes and outcomes after heart transplantation and African American and Caucasian American heart transplant recipient outcome after heart transplantation. We demonstrate that specific SNPs in African American and Caucasian American ethnic/racial groups can identify heart transplant patients at risk of poor outcome.

### Inflammatory Genes Provide a Signal to Identify High-risk Afam or Caam Heart Transplant Patients.

We used statically significant SNPs in linkage disequilibrium found within the Immunochip Verson 1 (V1) platform to develop risk groups within each sample group of African American and Caucasian American heart transplants. The sample was reclassified into two ethnic/racial groups based upon Principal Component analysis (Tian et al. European Population Genetic Substructure: Further Definition of Ancestry Informative Markers for Distinguishing among Diverse European Ethnic Groups. Molecular Medicine, 2009. 15(11-12): p. 371-383), which is incorporated herein by reference in its entirety as though fully set forth. All SNPs with a minor allele frequency (MAF)>1% and missing rate of <5% (and Hardy-Weinberg equilibrium (HWE) p=0.001) were included (102,647) in the analysis. Genotype clusters were manually reviewed to ensure correct allele calling. (Tables 1 and 2).

Within each ethnic/racial group, death is determined by Kaplan Meier survival analysis. All SNPs with p-values less than 10⁻⁴ were selected and used to determine the risk allele with coding of homozygote=2, heterozygote=1 and non-risk allele=0. The genetic risk score (GRS) was calculated by summing the risk alleles across the loci for each study participant within each ethnic/racial group. A group median score was determined and subjects were categorized as low risk or high risk relative to the group median. GRS in African American and Caucasian American Htx were determined independently. If the risk score is greater than the median, then the patient is considered high risk; if the risk score is less than or equal to the median, then the patient is considered low risk.

For example, the median GRS of each ethnic/racial group is used as the bar to determine high or low risk within that ethnic/racial group. For example, the median score for African American is 1.19 or about 1, and Caucasian American is 1.69 or about 2. A patient having GRS equal or below the group median score is at low risk, while a patient having GRS above the group median score is at high risk. We further explored the clinical context of these two heart transplant patient groups on select clinical variables of interest that can influence heart transplant outcomes. Significant differences between ethnic/racial groups were identified for recipient age, donor age, gender and disease type (Table 3).

We found that African American and Caucasian American heart transplant patients with low GRS had comparable survival (p=0.12; FIG. 3). For groups with high GRS score, AfAm patients had significantly lower survival compared to Caucasian American patients at 5 years (Log Rank p=0.022, HR 1.96 with 95% CI=1.09 to 3.52; FIG. 3). Using pathway analysis program Panther-db, SNPs from each ethnic/racial group were explored for their association with biological pathways. The only pathway that demonstrated overlap in both groups was the inflammation medicated by chemokines and cytokines. All other pathways were mutually exclusive to the ethnic/racial groups found in the above cohort of significant SNPs in Table 1 and Table 2 and Table 4.

TABLE 1
African American Significant SNPs

| | | Risk | Protective | Gene | Gene | | Exp- | |
|---|---|---|---|---|---|---|---|---|
| Chr | SNP | Allele | Allele | Symbol | Location | Coeff | coff | p-Value |
| 1 | rs12030062 | T | C | PLXNA2 \| | INTERGENIC | 2.25121 | 9.4992 | 0.000013274 |
| | | | | MIR205HG | | | | |
| 4 | rs2727438 | C | A | Chr4q34.3 | INTERGENIC | 2.56138 | 12.9537 | 0.000024558 |
| 10 | rs73266737 | C | T | CCNY | INTRON | 2.84468 | 17.196 | 0.000036576 |
| 15 | rs8032616 | A | C | SQRDL \| | INTERGENIC | 1.80108 | 6.0562 | 0.000007722 |
| | | | | SEMA6D | | | | |
| 15 | rs10519060 | G | A | SQRDL \| | INTERGENIC | 1.61536 | 5.0297 | 0.00007926 |
| | | | | SEMA6D | | | | |
| 17 | rs3785437 | T | G | MRPL38 \| | INTERGENIC | 3.31311 | 27.4704 | 0.000088496 |
| | | | | FBF1 | | | | |
| 17 | rs7221109 | T | C | CCR7 \| | INTERGENIC | 1.82547 | 6.2057 | 0.000077551 |
| | | | | SMARCE1 | | | | |
| 17 | rs62076937 | T | C | STAT5B | INTRON | 2.99377 | 19.9607 | 0.000072758 |
| 21 | rs2826929 | A | G | NCAM2 \| | INTERGENIC | 3.01247 | 20.3376 | 0.000046588 |
| | | | | LINC00317 | | | | |

TABLE 2
Caucasian American Significant SNPs

| | | Risk | Protective | Gene | Gene | | Exp- | |
|---|---|---|---|---|---|---|---|---|
| Chr | SNP | Allele | Allele | Symbol | Location | Coeff | coff | p-Value |
| 1 | rs6690278 | A | G | PLA2G4A | INTRON | 1.97015 | 7.1717 | 0.000083071 |
| 2 | rs2355570 | C | T | GLS | INTRON | 0.79828 | 2.2217 | 0.00005657 |
| 3 | rs115230839 | A | G | GLB1 | INTRON | 2.43167 | 11.3779 | 0.000007058 |
| 3 | rs17050452 | A | G | LOC642891 | INTERGENIC | 0.7731 | 2.1665 | 0.000092408 |
| 4 | rs7688988 | C | T | NFXL1 | INTRON | 1.3125 | 3.7155 | 0.000097161 |
| 4 | rs80165265 | T | G | KIAA1109 | INTRON | 1.91188 | 6.7658 | 0.00009726 |
| 7 | rs1991764 | G | A | ARL4A \| ETV1 | INTERGENIC | −0.91561 | 0.4003 | 0.000082208 |
| 8 | rs4922070 | A | G | CSGALNACT1 | INTRON | 0.83658 | 2.3085 | 0.0000351 |
| 12 | rs7957672 | C | G | VWF | INTRON | 1.80463 | 6.0777 | 0.000007327 |
| 12 | rs2544081 | T | G | ANO6, | INTERGENIC | 1.0186 | 2.7693 | 0.000039542 |
| | | | | LINC00938 | | | | |
| 16 | rs6564724 | G | A | Crh16q23.2 | INTERGENIC | 0.79589 | 2.2164 | 0.000095866 |
| 20 | rs111315210 | A | G | SERINC3 | INTRON | 1.79974 | 6.0481 | 0.000026388 |

TABLE 3
Demographics: Overall Group Comparisons

| | African | | Caucasian | | | |
|---|---|---|---|---|---|---|
| | American | | American | | | |
| Variable | (n = 53) | | (n = 205) | | P-Value | Test |
| Age | 53.4 ± 9.9 | | 57.7 ± 11.3 | | 0.012 | T-Test |
| Female | 21/53 | (39.6%) | 47/204 | (23.0%) | 0.022 | Fisher Exact |
| Diabetes Mellitus | 8/53 | (15.1%) | 65/205 | (31.7%) | 0.017 | Fisher Exact |
| Disease Type | | | | | <0.0001 | Fisher Exact |
| Idiopathic | 34/52 | (65.4%) | 61/203 | (30.0%) | | |
| Ischemic | 13/52 | (25.0%) | 128/203 | (68.1%) | | |
| Congenital | 0/52 | (0.0%) | 4/203 | (2.0%) | | |
| Other | 5/52 | (9.6%) | 10/203 | (4.9%) | | |
| Pre-Transplant | 1.5 ± 1.2 | | 1.4 ± 0.8 | | 0.580 | T-Test |
| Creatinine | | | | | | |
| Creatinine ≧1.5 | 19/50 | (38.0%) | 52/202 | (25.7%) | 0.113 | Fisher Exact |
| Creatinine ≧2.0 | 2/50 | (4.0%) | 24/202 | (11.9%) | 0.123 | Fisher Exact |
| PRA Max | 15.6 ± 25.1 | | 7.9 ± 16.5 | | 0.069 | Wilcoxon rank sum |
| PRA >10% | 14/47 | (29.8%) | 43/182 | (23.6%) | 0.450 | Fisher Exact |
| Ischemic Time | 163.0 ± 43.8 | | 173.1 ± 50.6 | | 0.180 | T-Test |
| Donor Age | 29.7 ± 12.9 | | 34.2 ± 12.4 | | 0.021 | T-Test |
| Female Donor | 11/53 | (20.8%) | 52/205 | (25.4%) | 0.590 | Fisher Exact |

TABLE 4

| Significant African | Significant Caucasian |
|---|---|
| American SNPs Pathways | American SNPs Pathways |
| EGF receptors signaling | Endothelin signaling |
| Inflammation mediated by | Oxidative stress response |
| chemokines and cytokines | |
| Interleukin signaling | Inflammation mediated by |
| | chemokines and cytokines |
| JAK/STAT signaling | Gonadotropin-releasing |
| | hormone receptor |
| PDGF signaling | CCKR signaling |
| | VEGF signaling |
| | Blood coagulation |

TABLE 5
Top Three Causes of Death between Ethnic/racial Groups

| African American Cause of Death | | Caucasian American Cause of Death | |
|---|---|---|---|
| Acute Rejection | 33.3% | Acute Rejection | 8.5%% |
| Graft Failure | 16.7% | Graft Failure | 10.5% |
| Infection | 33.3% | Infection | 8.5% |

TABLE 6
Demographics: High Risk African American Group
Comparisons to the Caucasian American Group

| | African | | Caucasian | | | |
|---|---|---|---|---|---|---|
| | American | | American | | | |
| Variable | (n = 26) | | (n = 113) | | P-Value | Test |
| Age | 53.0 ± 8.5 | | 57.5 ± 11.8 | | 0.069 | T-Test |
| Female | 5/26 | (19.2%) | 24/112 | (21.4%) | >0.999 | Fisher Exact |
| Diabetes Mellitus | 4/26 | (15.4%) | 36/113 | (31.9%) | 0.148 | Fisher Exact |
| Disease Type | | | | | 0.097 | Fisher Exact |
| Idiopathic | 13/25 | (52.0%) | 37/111 | (33.3%) | | |
| Ischemic | 9/25 | (36.0) | 65/111 | (58.6%) | | |
| Congenital | 0/25 | (0.0%) | 3/111 | (2.7%) | | |
| Other | 3/25 | (12.0%) | 6/111 | (5.4%) | | |
| Pre-Transplant | 1.3 ± 0.6 | | 1.4 ± 0.8 | | 0.810 | T-Test |
| Creatinine | | | | | | |
| Creatinine ≧1.5 | 8/24 | (33.3%) | 28/111 | (25.2%) | 0.450 | Fisher Exact |
| Creatinine ≧2.0 | 1/24 | (4.2%) | 12/111 | (10.8%) | 0.460 | Fisher Exact |
| PRA Max | 18.1 ± 28.1 | | 7.9 ± 17.2 | | 0.380 | Wilcoxon rank sum |
| PRA >10% | 7/21 | (33.3%) | 25.111 | (10.8%) | 0.420 | Fisher Exact |
| Ischemic Time | 163.0 ± 52.1 | | 177.5 ± 53.3 | | 0.210 | T-Test |
| Donor Age | 29.3 ± 11.7 | | 33.8 ± 12.2 | | 0.093 | T-Test |
| Female Donor | 2/26 | (7.7%) | 29/113 | (25.7%) | 0.065 | Fisher Exact |

TABLE 7
High Risk African American Potential
Predictors by Univariable Analysis

| Effect | DF | Score Chi-Square | Pr > Chi-Square |
|---|---|---|---|
| Gender | 1 | 0.2689 | 0.6041 |
| Ischemic Time | 1 | 7.4760 | 0.0063 |
| Diabetes Mellitus | 1 | 0.9869 | 0.3205 |
| Creatinine ≧1.5 | 1 | 1.6308 | 0.2016 |
| Disease Type | 1 | 0.0022 | 0.9623 |
| Donor Age | 1 | 0.3340 | 0.5633 |
| PRA Yes/No | 1 | 0.7460 | 0.3877 |

TABLE 8
High Risk Caucasian American Potential
Predictors by Univariable Analysis

| Effect | DF | Score Chi-Square | Pr > Chi-Square |
|---|---|---|---|
| Gender | 1 | 1.4946 | 0.2215 |
| Ischemic Time | 1 | 0.0020 | 0.9644 |
| Diabetes Mellitus | 1 | 0.8459 | 0.3577 |
| Creatinine ≧1.5 | 1 | 0.4307 | 0.5116 |
| Disease Type | 1 | 0.0108 | 0.9174 |
| Donor Age | 1 | 0.0013 | 0.9716 |
| PRA Yes/No | 1 | 0.4533 | 0.5008 |

TABLE 9
10-Year Cause of Death by Race High Risk Groups

| | Caucasian | | African | |
|---|---|---|---|---|
| | American | | American | |
| Cause of Death | (n = 47) | | (n = 18) | |
| Acute Rejection | 8.51% | (4) | 33.33%* | (6) |
| Cardiac Allograft Vasculopathy | 2.13%* | (1) | 0.00% | (0) |
| Graft Failure | 10.64% | (5) | 16.67%* | (3) |
| Infection | 8.51% | (4) | 33.33%* | (6) |
| Malignancy | 8.51% | (4) | 0.0% | (0) |
| Multi-Organ System Failure | 6.38% | (3) | 0.0% | (0) |
| Other (non-cardiac) | 44.68%* | (28) | 11.11% | (2) |
| Pulmonary | 6.38% | (3) | 5.56% | (1) |
| Renal Failure | 4.26% | (2) | 0.00% | (0) |

*p < 0.05

### Ethnicity Assignment by Principal Component Analysis.

Principal Component Method. In this study we utilized the Principal Component Analysis (PCA) to validate self-reported ethnicity (Tian et al. European Population Genetic Substructure: Further Definition of Ancestry Informative Markers for Distinguishing among Diverse European Ethnic Groups. Molecular Medicine, 2009. 15(11-12): p. 371-383), which is incorporated herein by reference in its entirety as though fully set forth). PCA is sensitive to the relative scaling of population variation based upon minor allele's distribution. This procedure assumes the frequency of the minor alleles as numeric or additive. As the PCA accounts for variations, subject groups differ from the minor allelic expected frequency model and become outliers. In utilizing the PCA refinement in our genetic risk scores study, three outliers in the African American group were eliminated. This statistical procedure is a common approach to address population differences [Patterson, N., A.L. Price, and D. Reich, Population Structure and Eigenanalysis. PLoS Genetics, 2006. 2(12): p. e190]. We validate that the candidate SNPs found in these data can be reproduced when analyzed in geographically distinct populations.

PCA is a commonly accepted statistical approach to assigning ethnicity. Allelic frequency variation is clustered in ethnic/racial groups and is associated with geographic origin and migration (Bryc et al., 2015, the Genetic ancestry of African Americans, Latinos and European American across the United States, American Journal of Human Genetics, 96:37-53; Salas et al., 2005, Charting the ancestry of African Americans, Am J Hum Genet, 77(4): p.676-80). These patterns between ethnic/racial groups may provide a mechanistic basis for the differences in inflammatory SNPs that were associated with poor outcomes in both groups comparing high to low genetic risk scores but worse outcomes overall when comparing ancestry-specific SNPs of African Americans to Caucasian Americans heart transplant recipients. Principal component analysis, a mathematical algorithm, annotates complex genetic data (SNPs from two ethnic/racial groups) and converts the data to small categories by calculating the main axes or “principal components” (PCs) of variation. These components are orthogonal vectors that capture the maximum variability present in the data. The first component explains the most variation in the data, and each subsequent component accounts for another, smaller part of the variability. When applied to genotype data, these axes of variation have been shown to have a striking relationship with geographic origin (Visscher et al., 2009, Application of principal component analysis to pharmacogenomic studies in Canada, the Pharmacogenomics Journal, 9:362-272). To adjust for population substructures and correct for population stratifications among different ethnic/racial groups, we first calculate the covariance matrix with the genetic data, and then calculate the principal components with singular value decomposition. Top four (usually 4-10) principal components are then used for SNP identification with a multivariate regression model. A generic regression model with 4 principal components (PCs) is as follows:

Y=F(β₀+β₁PC₁+β₂PC₂+β₃PC₃+β₄PC₄+β₅G),

in which G is a SNP to be tested. Cox regression is used for identifying survival-associated SNPs, while logistic regression is used for case-control study.
Ethnicity Assignment by Mitochondrial DNA (mtDNA) Haplotypes.

Mitochondrial DNA (mtDNA). Mitochondria, the energy-producing organelles present in all cells (except red blood cells) possess their own genome of 23,000bp, and there are over a thousand copies of mtDNA per cell. There can be minor variations in the mtDNA sequence, and allelic variations in the mitochondrial genome can be present within the same cell, a condition referred to as heteroplasmy. The inheritance of mtDNA is passed from mother to daughters and sons [Jackson, F. L., Human genetic variation and health: new assessment approaches based on ethnogenetic layering. Br Med Bull, 2004. 69: p. 215-35]. HapMap worldwide mtDNA database is widely used to assess ancestral origin based on conserved polymorphisms and is applicable to African American AA mtDNA haplotypes. Forced migration of Africans brought to North America during the Atlantic Slave trade originated from either West or Central Africa. mtDNA sequences were studied in a sample of 1148 African American residing in United States. These researchers confirmed the African geographic origins as 55% from West Africa and 41% from West Central and South Africa [Salas, A., et al., Charting the ancestry of African Americans. Am J Hum Genet, 2005. 77(4): p. 676-80]. Polymorphisms in coding and noncoding regions of the mitochondrial genome are common and an increasing number of polymorphisms are associated with various human diseases ranging from cancer and heart failure to schizophrenia and diabetes [Strauss, K. A., et al., Severity of cardiomyopathy associated with adenine nucleotide translocator-1 deficiency correlates with mtDNA haplogroup. Proc Natl Acad Sci USA, 2013. 110(9): p. 3453-8]. While in this study uses mtDNA to assign ancestry (by itself or in conjunction with self-report and/or Principal Component Method), we recognize that it is possible to investigate the linkage between mtDNA polymorphisms and rejection severity and outcome. Genomic and mtDNA are applied to the mtDNA haplogroup analysis SNP chip in order to assign ancestry [Royal, C.D., et al., Inferring genetic ancestry: opportunities, challenges, and implications. Am J Hum Genet, 2010. 86(5): p. 661-73]. Sequencing studies have shown that analysis of the hypervariable regions HVR1 and HVR2 can be used to assign ethnicity; these are noncoding regions in the D-loop of mtDNA [Lee et al. Inferring ethnicity from mitochondrial DNA sequence. BMC Proc, 2011. 5 Suppl 2: p. S11, which is incorporated herein by reference in its entirety as though fully set forth]. For example, the haplogroup L is associated with African Americans and the haplogroup H is associated with Caucasian Americans.

Validate the Self-reported Ethnicity Derived from Afam and Caam Heart Transplant Recipients Derived from Two Geographic Regions

The overall percentage of African American AfAm Htx in our program is 12%, corresponding closely to the 14% African American proportion in the general population. To expand the sample size and geographic areas, we obtain samples from two large Htx centers that are combined with samples from Cedars Sinai Medical Center in Los Angeles California. Each of the two centers contributes 75 African American and 100 Caucasian American DNA samples along with specific clinical data. The total samples for analysis are 150 African American and 200 Caucasian American combined with 75 African American and 204 CaAm from Cedars-Sinai Medical Center for a total of 629 samples. We identified these Htx programs as they have sufficient numbers of archived DNA samples and appropriate clinical demographic data on their African American AfAm and CaAm heart transplant patients. Our findings address the disparity in survival experienced by AfAm Htx recipients' post-heart transplantation. Our method increases reliability and lays the foundation for identifying high genetic risk patients.

Our findings were validated and enhanced upon the use of PCA to determine population ethnic/racial ancestry. This is an acceptable approach; however, utilizing mtDNA provides a second validation and enhancement of ethnicity to support individualized medicine. We repeat the PCA approach as a standard validation of ancestry but also utilize mitochondrial DNA (mtDNA) as a second approach to establish ancestry. Polymorphisms in the mitochondrial genome are common and an increasing number of polymorphisms have been shown to be associated with various human diseases ranging from cancer and heart disease and transplantation to schizophrenia and diabetes. While using mtDNA to assign ancestry (by itself or in conjunction with self-report and Principal Component Method), we also investigate the linkage between mtDNA polymorphisms and rejection severity and outcome within groups.

Samples of mtDNA are isolated using the Qiagen Miniprep Kit (Germantown, Pa.) per manufacturer to produce eluted DNA in 100uL of elution buffer. The mtDNA sample is purified. Beads are added in proportion by volume on a magnetic stand and washed twice with ethanol. The beads are air dried and mtDNA is eluted and re-suspended in TE buffer. Enrichment of mtDNA occurred using primers and real time PCR.

We may see some discordance between PCA, which uses nuclear DNA markers, and mtDNA ethnic/racial determinations, because mtDNA is maternally transmitted. Thus offspring of a CaAm mother would have CaAm mtDNA markers yet PCA might indicate AfAm ancestry. While such instances are likely to be relatively infrequent, we treat these individuals as AfAm. In some instances, mtDNA may modify survival outcome post heart transplantation in either ethnic/racial group and by GRS.

Identify Genes in Linkage Disequilibrium with Snps Confirmed by the Immunochip Platform and Stratify these Snps into High/low Genetic Risk Groups for Poor Heart Transplant Outcome

Immunochip V1 was used for the study. We also run all samples (total of 629 from 3 centers) including the previously-obtained samples using the new version Immunochip V2 probes. Once the candidate SNPs are identified, we utilize the same protocol as for Immunochip V1. Running all samples under the same conditions controls for consistency. The newly available SNPs on the Immunochip V2 probe platform present an unprecedented expansion of opportunities for the analysis of genetic variation and function.

Our findings were significant in illuminating genetic markers associated with poor outcome in African American AfAm Htx patients. We also examine clinical data on all patients to describe the clinical presentation of these high risk recipients. SNP analysis uses the Immunochip V2 genotyping array made by Illumina per manufacturer's protocol. Our data was analyzed using Immunochip V1 which contained 192,403 SNPs covering chromosome 1-22. The new Immunochip V2 platform has eliminated 16,472 SNPs and added 98,000 new SNPs for a total of 270,931 SNPs across chromosomes 1-22. First, we proceed to the concordance procedure, in which all samples are analyzed at one time using the Immunochip V2 according to Illumina protocol. In the Immunochip V2 run, we include 4 DNA samples from the V1 study as positive controls: These samples represent AfAm hi-risk, AfAm low-risk, CaAm hi-risk, and CaAm low-risk. Secondly, we compare the SNPs of interest from both ethnic/racial groups (African American African American 9 and Caucasian American 12) identified from the study SNP ID number between Immunochip V1 and V2. If the SNPs are not detected on the V2 chip, a proxy SNP that is in 100% R2=1.0 proximity is chosen. If a perfect proxy SNP is not found, a proxy SNP within R2=0.8 proximity is chosen. If appropriate SNPs are not found, all missing SNPs are excluded from analysis. Lastly, significant new SNPs with p-Values less than (10⁻⁴) from the Immunochip V2 in linkage disequilibrium are included in the genetic risk score stratification of risk assignment.

This study makes a major contribution for a particularly vulnerable patient population that has received a scarce organ. No study to date has utilized the genetic risk score approach to stratify patients into high risk groups. Our approach provides strategies to overcome the currently dismal survival in AfAm patients receiving a heart transplant.

We expand our analysis to determine if there are additional SNPs identified from the Immunochip V2 that can contribute to a genetic risk score for HTx patients of African American or Caucasian American ancestry. There is a robust inclusion of new inflammatory SNPs that further inform pathways. These new findings from this larger geographic sample can explain the differences seen between our groups of interest. Determination of the GRS for this study follow the procedure described herein. We stratify both ethnic/racial groups (African American and Caucasian American) by high and low risk. Our study both validates our GRS procedure and expands the pathways associated with our SNPs of interest. This study provides the bases for mechanistic investigations to support individualized approaches to post transplant management.

These studies utilize the Immunochip V2. We confirm our findings and run a small test set with previously analyzed DNA samples before running the full cohort. Our study leads to identification of additional SNPs associated with Htx outcome; here, bioinformatics analysis is essential to understand the importance of these new SNPs. In addition, we can investigate the Affymetrix platform microarray for our SNPs of interest.

Explore the Impact of mtDNA Variation on Survival Outcomes Comparing AfAm and CaAm Heart Transplant Recipients

The mitochondrial genome is subject to greater variation because of its limited DNA repair mechanisms. It has been suggested that mtDNA evolution has enabled human adaptation to different environments through shifting the metabolic milieu and as a result, altering the epigenetic marks on nuclear DNA and thus the transcriptome. Moreover, mtDNA variation has a profound impact on disease penetrance; for instance, in patients with ANTI mutations, the severity of cardiomyopathy is tightly related to mtDNA haplogroups. mtDNA haplogroup is also associated with exercise capacity after endurance training in humans, suggesting that changes in the DNA control region (which varies across haplogroups) might influence mitochondrial biogenesis needed for endurance capacity [Murakami, H., et al., Polymorphisms in control region of mtDNA relates to individual differences in endurance capacity or trainability. Jpn J Physiol, 2002. 52(3): p. 247-56]. Mitochondrial efficiency in skeletal muscle could impact cardiac workload and eventual outcome, but it does not explain a link to inflammation. However, mtDNA is recognized by receptors for damage-associated molecular patterns (DAMPs) that activate innate immunity leading to production of various cytokines. Thus, without wishing to be bound by any particular theory, we believe that alterations in mitochondrial function (related to mtDNA haplotypes or SNPs) may alter the threshold for an inflammatory response. Without wishing to be bound by any particular theory, we believe that some mtDNA genomes result in mitochondria that are intrinsically more vulnerable to stress and thus more easily release mtDNA into the cytoplasm where it can trigger receptors for DAMPS (e.g. TLR9, NLRP3), leading to cytokine release. If individuals with these vulnerable mitochondria also have nuclear-encoded polymorphisms associated with increased cytokine activity, they would be expected to have an exaggerated response to injury. While we can perform complete mtDNA sequencing in all patients, we first perform mtDNA haplogroup and SNP analysis.

We determine whether mtDNA haplogroups or SNPs can be linked to Htx outcome. In our data the top three causes of death were unequal among the African American AfAm and Caucasian American heart transplant recipients. A greater incidence of acute rejection, graft failure and infection was associated with African American AfAm's in the high genetic risk group compared to Caucasian American heart transplant recipients. Without wishing to be bound by any particular theory, the high incidence of infection in the African American group was likely related to increased immunosuppression to treat acute rejection episodes. In the study, we investigate if the presence of mtDNA SNPs are the same or different in the high genetic risk group. Such a marker can provide clinicians with a mechanism for risk stratification and individualized treatment approaches.

Determining the existence of significant mtDNA haplogrops within the ethnically separate genetic high risk group can provide the clinicians with markers to further identify and stratify high risk for poor outcomes. Such predictive markers can become targets to improving survival in these patients.

We determine whether the recipient's mtDNA haplogroups or SNPs act as disease modifiers. In the study, for the AfAm in the high genetic risk group less than 10% were alive at 10 years compared to 80% AfAm who were in the low risk group. The question arises as to whether presence of specific mtDNA haplotypes is protective and is associated with low genetic risk score and modifies outcome. We explore the presence of these associations on survival.

This study generates evidence to support the hypothesis that mtDNA variations can modify outcome in Htx patients. While the study first uses haplogroup and SNP analysis, we conduct a subsequent investigation involving sequencing. Importantly, since almost the entire mitochondrial genome encodes either tRNAs, rRNAs, or proteins (no introns or non-coding stretches), almost any mutation/variant may result in a change at the protein level. In subsequent analysis, we focus on SNPs that result in a change in the amino acid, particularly ones that might alter protein function (e.g., a change from a hydrophobic amino acid to a polar one). The findings can elucidate the relationship between mtDNA, mitochondrial function, and activation of innate immunity.

### Statistical Analysis

Sample Size Calculation: we validate 21 known SNPs we identified in our study. A two-sided log rank test is used for sample size calculation. Bonferroni correction is utilized to counteract the problem of multiple comparisons. With statistical significance level of 0.05/21=0.0024, an overall sample size of 629 subjects (404 Caucasian American and 225 African American) achieves 80.0% and 90% powers to detect the hazard ratios of 1.41 and 1.47, respectively, when the hazard ratio of CA group is set to 1.00.

Data Analysis: after data from ImmunoChip version2 is processed with SAM tools for SNP calling, we first validate the 21 candidate SNPs with the newly generated data. In addition, Kaplan-Meier curves, log rank statistical tests, and Cox regression is used to identify new mutations associated with survival outcomes of heart transplantation. The assumption of proportional hazard ratio of Cox regression is tested. Novel SNPs associated poor post-heart transplant outcomes and racial disparities are also identified for further studies. These newly identified SNPs are further studied at the gene level through annotation and pathway analysis. Pathways and related molecular functions are explored through gene enrichment analysis.

The key innovation of this study is the concept of developing a genetic risk score that identifies heart transplant patients who are at risk for early death. We believe that this study validates the approach of stratifying high risk Htx patients according to inflammatory SNPs. Given that inflammatory mediators underpin rejections, our work focuses upon clinical trials targeting this high risk group. This study also demonstrated that mtDNA haplotypes could act as disease modifiers, exacerbating (or nullifying) the inflammatory gene SNPs associated with poor Htx outcome.

We confirm the SNPs found in the ethnic/racial group of interest from the study, and we further investigate the genetic pathways and mechanism mediating the transplant outcomes. We show the role of mtDNA as modifier for disease outcomes in the ethnic/racial groups of interest, and further investigate the interaction of nDNA and mtDNA and the role of immune modulation after heart transplantation.

The polymorphism imm_3_33039496 is located at the position No. 33039496 on Chromosome 3. The polymorphism seq-VH-2664 is located at the position No. 123475199 on Chromosome 4. The polymorphism imm_20_42579594 is located at the position No. 42579594 on Chromosome 20.

### Example 3

### Genetic Risk Score (GRS) Predicts Worse Survival in African American Heart Transplant Recipients at 10 Years

Ethnic/racial heart transplant outcomes are disparate when comparing African Americans and Caucasian Americans. Large scale analysis of the UNOS database including race using a case-control method or era effects analysis have demonstrated worse ten year survival in African Americans post heart transplantation. Exploration of the UNOS database of 20,000 donor to recipient race-matched heart transplants and found a 46% increase in the cumulative risk of death at 6 months and 6 years in African Americans compared to Caucasian Americans.

When exploring cause of death post heart transplantation, African Americans (AA) are more likely to die of cardiovascular events such as rejection and/or graft failure compared to death caused by cancer or infection found in Caucasian Americans. The basis for increased risk of CV death in AA HTx recipients may be due to higher prevalence of immune-genetic risk which could mediate rejection by inflammation and variable drug metabolism. However, the exact role of immune-genetics in contributing to survival post heart transplantation has not been established.

We investigate the impact of inflammatory single nucleotide polymorphisms (SNPs) on development of a genetic risk score (GRS) predictive of survival in AA and CA heart transplant recipients from our institution, and we explore clinical phenotype predictors of survival within the genetic risk categories and ethnic/racial groups.

We analyzed 257 heart transplant recipients (AA 53 and CA 204) with DNA samples banked in the HLA laboratory between 2000 and 2011. Principle Component Analysis was used to confirm/reclassify self-reported ethnicity (Tian et al. European Population Genetic Substructure: Further Definition of Ancestry Informative Markers for Distinguishing among Diverse European Ethnic Groups. Molecular Medicine, 2009. 15(11-12): p. 371-383), which is incorporated herein by reference in its entirety as though fully set forth).

Immunochip V1 Illumina Infinium SNP microarray probes platform was used to identify significant inflammatory genes used to create a genetic risk score. Genetic risk score (GRS) included all SNPs with minor allele frequency (MAF)>1% /missing rate<5% (HWE p=0.001) were included (102,647) in the analysis. GRS was determined based upon the presence of the risk allele. Each SNP risk allele was coded/assigned a value: Homozygote=2, Heterozygote=1, and Non-risk homozygote alleles=0. GRS was calculated by summing the risk alleles across the loci for each study participant within each ethnic/racial group. Based on the GRS, subjects were classified into high risk groups determined by the GRS being>the median score and low risk groups being GRS≦the median score.

Endpoints of the study included 10-year survival estimated by Kaplan-Meier method. Univariable and Multivariable analyses were performed for multiple risk factors. Cox proportional hazards models were used to assess factors related to 5-year survival and to obtain hazard ratios and their 95% confidence intervals 5-year survival estimated by Kaplan-Meier method.

Principle component analysis (PCA) statistically determined ethnicity. Significant inflammatory SNP's independently associated with African American (9) and Caucasian Americans (12) distinguished between patients at high genetic risk or low genetic risk for poor survival. Within the high GRS group, African Americans versus Caucasian Americans had worse outcome at 10 years post heart transplant which was associated with increased rejection, infection and graft failure as cause of death. No difference in survival was demonstrated in the low GRS African American group compared to the Caucasian American group.

The use of a genetic risk score as a measure of vulnerability to risk stratify high risk patients for poor outcomes after heart transplantation is useful for patient care. Our data have identified candidate SNPs offering an approach to exploring the disparity among ethnic/racial groups where appropriate intervention may be possible.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method, comprising:
obtaining a sample from the subject;
assaying the sample to detect the risk alleles at one or more SNPs;
calculating a genetic risk score (GRS) of the subject based on the detected risk alleles at the one or more SNPs;
determining that the subject has an increased likelihood of poor prognosis if the GRS of the subject is above the mean or median GRS of the sample population or determining that the subject has an increased likelihood of good prognosis if the GRS of the subject is the same as or above the mean or median GRS of the sample population; and selecting a therapy if poor prognosis is determined.

2. The method of claim 1, wherein the subject has end-stage heart failure or a severe coronary artery disease.

3. The method of claim 1, wherein the subject is waiting for a heart transplant or has received a heart transplant.

4. The method of claim 1, wherein the GRS is the total number of the detected risk alleles at the one or more SNPs.

5. The method of claim 1, wherein the subject is of African ancestry.

6. The method of claim 5, wherein the one or more SNPs comprise one, two, three, four, five, six, or more, or all of: rs12030062, rs2727438, rs73266737, rs8032616, rs10519060, rs3785437, rs7221109, rs62076937, and rs2826929.

7. The method of claim 6, wherein the GRS of the subject is above the median or mean GRS of the population of African ancestry and the heart transplant is prognosed with a poor clinical outcome.

8. The method of claim 7, wherein the subject is prognosed with a poor heart transplant and the selected therapies comprise heart transplant and administration of an effective amount of an IL-6 inhibitor, a JAK-STAT inhibitor or combinations thereof, wherein the inhibitors are administered before, during and/or after the heart transplant.

9. The method of claim 7, wherein the subject is prognosed with a good heart transplant and the selected therapies comprise heart transplant.

10. The method of claim 4, wherein the subject is of European ancestry.

11. The method of claim 10, wherein the one or more SNPs comprise one, two, three, four, five, six, or more, or all of: rs6690278, rs2355570, rs115230839, rs17050452, rs7688988, rs80165265, rs1991764, rs4922070, rs7957672, rs2544081, rs6564724, and rs111315210.

12. The method of claim 11, wherein the GRS of the subject is above the median or mean GRS of the population of European ancestry, and the heart transplant is prognosed with a poor clinical outcome.

13. A method of classifying a subject with a cardiovascular condition, comprising:
obtaining a sample from the subject;
assaying the sample to detect the risk alleles at one or more SNPs;
calculating a genetic risk score (GRS) of the subject based on the detected risk alleles at the one or more SNPs; and
classifying the subject into a group based on the GRS of the subject.

14. The method of claim 13, wherein the cardiovascular condition is end-stage heart failure or a severe coronary artery disease.

15. The method of claim 13, wherein the GRS of the subject is not above the median or mean GRS of the population of the same ancestry as the subject, and the subject is classified into a low risk group.

16. The method of claim 13, wherein the GRS of the subject is above the median or mean GRS of the population of the same ancestry as the subject, and the subject is classified into a high risk group.
